(19) United States
(12) Patent Application Publication
(10) Pub. No.: US 2025/0304973 A1
(43) Pub. Date: Oct. 2, 2025

(54) HIGH-AFFINITY THROMBIN APTAMERS AND METHODS OF USE

(71) Applicant: Ayass Bioscience LLC, Frisco, TX (US)

(72) Inventors: Mohammad Ammar Ayass, San Angelo, TX (US); Lina Abi Mosleh, Frisco, TX (US)

(21) Appl. No.: 19/046,777

(22) Filed: Feb. 6, 2025

Related U.S. Application Data
(60) Provisional application No. 63/550,184, filed on Feb. 6, 2024.

(51) Int. Cl. C12N 15/115; A61P 7/02; C12N 15/10

(52) U.S. Cl. CPC C12N 15/115; A61P 7/02; C12N 15/1048; C12N 2310/16; C12N 2310/3515; C12N 2320/35

(57) ABSTRACT

Disclosed are aptamers, aptamer antidotes, and their use in the treatment of thrombosis and other thrombotic conditions.

This Application claims the benefit of U.S. Application No. 63/550,184, filed on Feb. 6, 2024, which is incorporated herein by reference in its entirety.

### I. REFERENCE TO SEQUENCE LISTING

The sequence listing submitted on May 19, 2025, as an .XML file entitled “11050-012US1_ST26.xml” created on Jun. 20, 2025, and having a file size of 33,943 bytes is hereby incorporated by reference pursuant to 37 C.F.R. § 1.52(e)(5).

### II. BACKGROUND

1. Thrombosis is a serious condition in which a blood clot forms inside a blood vessel (artery or vein). If thrombosis is untreated, the clot can slow, block, or travel in the blood flow, causing life-threatening emergencies such as thromboembolic stroke, heart attack, venous thromboembolism (VTE), or pulmonary embolism (PE). It is estimated that 900,000 Americans are affected by VTE each year and that 100,000 Americans die from PE. In recent years, the treatment of thrombosis has been gradually changing from conventional anticoagulants (heparin, vitamin K antagonists) to direct oral anticoagulants (DOACs) (oral direct thrombin inhibitors (DTIs), oral factor Xa inhibitors). In comparison to conventional anticoagulant therapies, DOACs have the advantages of oral administration, no need for frequent monitoring and dose adjustment, and fewer medication and food interactions.

2. Among the activated coagulation proteinases, thrombin is a unique coagulation protein that plays a central orchestrating role in procoagulation, anticoagulation, and platelet activation. In procoagulation, thrombin converts fibrinogen into insoluble fibrin clots while activating platelets and factors XIII, V, VIII, and XI. In anticoagulation, thrombin plays a regulatory and control role in the coagulation cascade by activating protein C, which together with its cofactor protein S degrades FVIIIa and FVa. The multiple roles of thrombin are mostly attributed to its highly dynamic three-dimensional molecular structure. Thrombin has a catalytic site and two anion-binding active sites, exosite I and exosite II. Exosite I is the active site of thrombin, and is responsible for interacting with various molecules, including fibrinogen, fibrin, heparin cofactor II, and protease-activated receptor. Exosite II is the specific site responsible for the activation of factors V factor VIII, as well as for heparin binding.

3. Dabigatran etexilate (Pradaxa) is an FDA-approved direct and reversible thrombin inhibitor. It is an orally administered anticoagulant specifically targeting thrombin, and has been approved for various indications, including prevention of systemic embolism in non-valvular atrial fibrillation, prevention of recurrent venous thromboembolism (VTE), and prevention of stroke. Dabigatran is an active form of dabigatran etexilate that competitively and selectively binds thrombin, preventing the conversion of soluble fibrinogen to insoluble fibrin and inhibiting thrombin-induced platelet aggregation. It is a fast-acting drug and can be rapidly reversed with idarucizumab, a monoclonal antibody that binds to dabigatran and neutralizes its anticoagulant properties. In the past decade of clinical application, research has found that dabigatran shares the same side effects, drug interactions, and limitations as DOACs. Taking dabigatran may cause stomach discomfort, bloody stools, pain or a burning sensation in the throat, etc.. Moreover, dabigatran should be avoided/is contraindicated in patients with creatine clearance (CrC1<30 mL/min), pregnancy, mechanical heart valves, and/or a history of serious dabigatran hypersensitivity. Furthermore, due to significant drug interactions there are over 150 medications that are not recommended for use with dabigatran (e.g., Abciximab, Capmatinib, Dalteparin, etc.). Finally, taking dabigatran increases the risks of bleeding, which could potentially be fatal in certain cases. Recent studies have found that there are no differences between DOACs and conventional anticoagulants in terms of their safety and long-term efficacy in preventing recurrent PE/VTE, mortality, and major bleeding. Therefore, development of effective and safe DOACs remains an ongoing challenge in the context of reducing both short-term and long-term negative outcomes associated with thrombosis. What is needed are new direct oral anticoagulants that overcome these obstacles.

### III. SUMMARY

4. Disclosed are aptamers, aptamer antidotes and methods of their use in the treatment of thrombosis and related conditions.

5. In one aspect disclosed herein are isolated nucleic acids (aptamers) comprising the sequence as set forth in SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21, or any fragment or variant thereof comprising at least 90% sequence identity thereto. In one aspect, the isolated nucleic acid can be in a pharmaceutical composition (such as, for example, an active component in a pharmaceutical composition).

6. Also disclosed herein is the RNA equivalent of any of the isolated nuclei acids (i.e., aptamers) of any preceding aspect.

7. In one aspect, disclosed herein are isolated nucleic acids of any preceding aspect, wherein the nucleic acid (i.e., aptamer) further comprises a detectable tag (including, but not limited to a latex bead, magnetic bead, fluorescence label; fluorescent probe, chemiluminescent labels, radiolabels, and/or nanoparticle probe) and/or a stabilizing moiety (including, but not limited to polyethylene glycol (PEG), triethylene glycol (TEG), or cholesterol) (such as, for example, SEQ ID NO: 16, SEQ ID NO: 17, SEQ ID NO: 18, SEQ ID NO: 19, SEQ ID NO: 20, SEQ ID NO: 21).

8. Also disclosed herein are kits comprising the isolated nucleic acid (i.e., aptamer) of any preceding aspect. For example, disclosed herein are kits comprising one or more of SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, and/or SEQ ID NO: 21. In some aspects, the kit may further comprises an aptamer antidote (such as, for example, SEQ ID NO: 23 and/or SEQ ID NO: 24). Thus, in one aspect, disclosed herein are kits wherein the isolated nucleic acid (i.e., the aptamer) comprises SEQ ID NO: 2, SEQ ID NO: 16, SEQ ID NO: 17, and/or SEQ ID NO: 18 and the aptamer antidote comprises SEQ ID NO: 23 or wherein the isolated nucleic acid (i.e., aptamer) comprises SEQ ID NO: 4, SEQ ID NO: 19, SEQ ID NO: 20, and/or SEQ ID NO: 21 and the aptamer antidote comprises SEQ ID NO: 24.

9. In one aspect, disclosed herein are methods of treating, inhibiting, decreasing, reducing, and/or ameliorating thrombosis or a thrombotic condition (including, but not limited to such as thromboembolic stroke, ischemia, heart attack, arterial thrombosis, deep vein thrombosis (DVT), disseminated intravascular coagulation (DIC), venous thromboembolism (VTE), central venous sinus thrombosis, cavernous sinus thrombosis, central retinal vein occlusion, branch retinal vein occlusion, Paget-Schroetter disease, Budd-Chiari syndrome, splanchnic venous thrombosis, renal vein thrombosis, ovarian vein thrombosis, jugular vein thrombosis, or pulmonary embolism (PE)), or the symptoms thereof in a subject in need thereof comprising administering to the subject one or more of the isolated nucleic acids (i.e., aptamers) of any preceding aspect. For example, disclosed herein are methods of treating, inhibiting, decreasing, reducing, and/or ameliorating thrombosis or a thrombotic condition (including, but not limited to such as thromboembolic stroke, ischemia, heart attack, arterial thrombosis, deep vein thrombosis (DVT), disseminated intravascular coagulation (DIC), venous thromboembolism (VTE), central venous sinus thrombosis, cavernous sinus thrombosis, central retinal vein occlusion, branch retinal vein occlusion, Paget-Schroetter disease, Budd-Chiari syndrome, splanchnic venous thrombosis, renal vein thrombosis, ovarian vein thrombosis, jugular vein thrombosis, or pulmonary embolism (PE)), or the symptoms thereof in a subject in need thereof comprising administering to the subject one or more isolated nucleic acids (aptamers) comprising the sequence as set forth in SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21.

10. Also disclosed herein are aptamer antidotes comprising the sequence the reverse complement of any isolated nucleic acid of any preceding aspect (such as, for example, SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21). In one aspect, the aptamer antidote comprises the sequence as set forth in SEQ ID NO: 23 or SEQ ID NO: 24.

11. In one aspect, disclosed herein are methods of treating, inhibiting, decreasing, reducing, and/or ameliorating an adverse event from administration of an aptamer therapy comprising administering to a subject having previously received the aptamer therapy, the aptamer antidote of any preceding aspect (such as, for example SEQ ID NO: 23 or SEQ ID NO: 24).

12. Also disclosed herein are methods of treating, inhibiting, decreasing, reducing, and/or ameliorating an adverse event from administration of an aptamer therapy wherein the aptamer comprises SEQ ID NO: 2, SEQ ID NO: 16, SEQ ID NO: 17, and/or SEQ ID NO: 18 and the aptamer antidote comprises SEQ ID NO: 23; or wherein the aptamer comprises SEQ ID NO: 4, SEQ ID NO: 19, SEQ ID NO: 20, and/or SEQ ID NO: 21 and the aptamer antidote comprises SEQ ID NO: 24.

### IV. BRIEF DESCRIPTION OF THE DRAWINGS

13. The accompanying drawings, which are incorporated in and constitute a part of this specification, illustrate several embodiments and together with the description illustrate the disclosed compositions and methods.

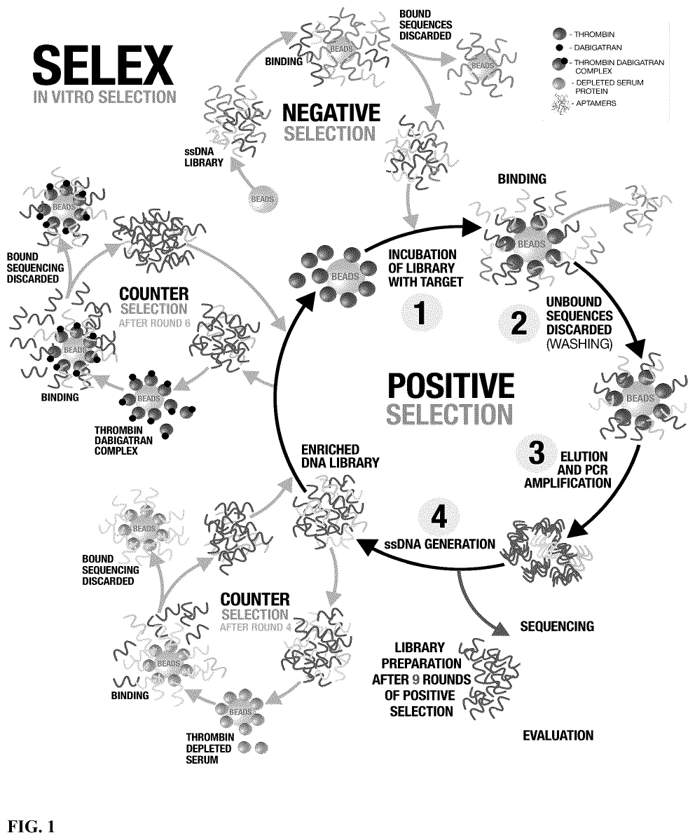

14. FIG. 1 shows an illustration of the SELEX procedure used to develop the human alpha-thrombin-specific aptamers. A library of single-stranded DNA oligonucleotides (10¹⁵ different unique sequences) was used. Each unique sequence contains random bases (40 nt) flanked by two conserved primer binding sites, which are used for PCR amplification. Negative selection was performed using bare magnetic beads (no thrombin) to remove nonspecific interactions between DNA oligonucleotides and magnetic beads. Human alpha-thrombin native protein was immobilized on NHS-Activated Magnetic Beads. In the first positive selection step, the library was incubated with human thrombin protein immobilized on NHS-activated magnetic beads (1), the unbound sequences were separated from the bound ones (2), and target-bound sequences were eluted from target molecules and amplified by PCR using biotinylated reverse primer (3). The PCR product was pulled down using streptavidin beads, and the specific single stranded DNA was separated using sodium hydroxide buffer and utilized in the next round of selection (4). The process was repeated for nine rounds with increasing selection stringency by increasing the stringency of the washing buffer to 0.5 M NaCl and increasing the washing time. To enhance the specificity of the selected oligonucleotides, counter-selection was performed after the fourth round using thrombin depleted serum proteins immobilized on magnetic beads. A second counter-selection was performed after the sixth round to enrich the library with aptamers for the active site of thrombin. We used dabigatran, a drug that binds directly to active site of thrombin. Dabigatran (5 nmol) was applied to thrombin beads to block the thrombin active site. ssDNA from the sixth SELEX round were loaded onto the beads and the flow-through was collected, followed by three further rounds of traditional selection.

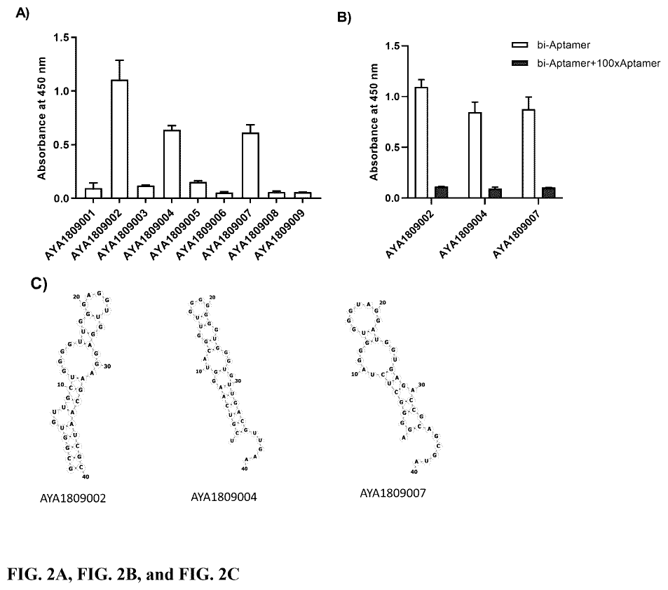

15. FIGS. 2A, 2B, and 2C show the binding of aptamers to thrombin. FIG. 2A shows the binding of the top nine enriched aptamer sequences to thrombin protein: thrombin protein was immobilized on a 96-well plate (see materials and method); the top nine enriched aptamers from the Next Generation Sequencing results were synthesized with a 3′ biotin group and incubated with the immobilized thrombin in duplicates. Absorbance was measured after incubation with streptavidin horseradish peroxidase bound to the biotinylated aptamer in the presence of the TMB substrate. Each bar shows the average of duplicate measurements. FIG. 2B shows a competition assay for aptamer binding to thrombin with an excess of non-biotin labelled aptamers. The specific binding of four aptamers that displayed binding to thrombin was tested in the presence of a 100-fold excess of the corresponding nonlabeled aptamer. All three aptamers show specific binding to thrombin that can be competed out in the presence of nonlabeled aptamer. Each bar shows the average of duplicate measurements. FIG. 2C shows the secondary structure prediction for selected aptamers AYA1809002 (SEQ ID NO: 2), AYA1809004 (SEQ ID NO: 4), and AYA1809007 (SEQ ID NO: 7).

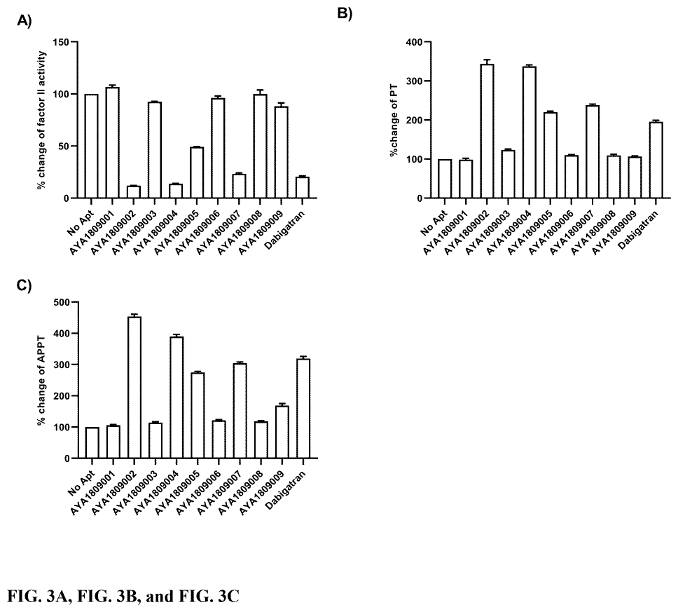

16. FIGS. 3A. 3B, and 3C show the effect of enriched aptamers on Factor II activity, Prothrombin time (PT), and Activated Partial Thromboplastin Time (APTT). Human plasma was incubated in the absence or presence of 2 mM of either Pradaxa or thrombin aptamers for 2 h at room temperature. (3A) Inhibition of Factor II Activity by thrombin aptamers as compared to dabigatran was determined by measuring Factor II activity in citrated plasma. Increase of PT time (3B) and APTT time (3C) by thrombin aptamers as compared to dabigatran was measured in human citrated plasma. All measurements were conducted using an ACLTOP coagulation analyzer manufactured by Instrumentation Laboratory. The relative activity of Factor II, PT, and APTT time was determined by comparing the treated plasma sample to the untreated one. Each bar shows the average of duplicate measurements.

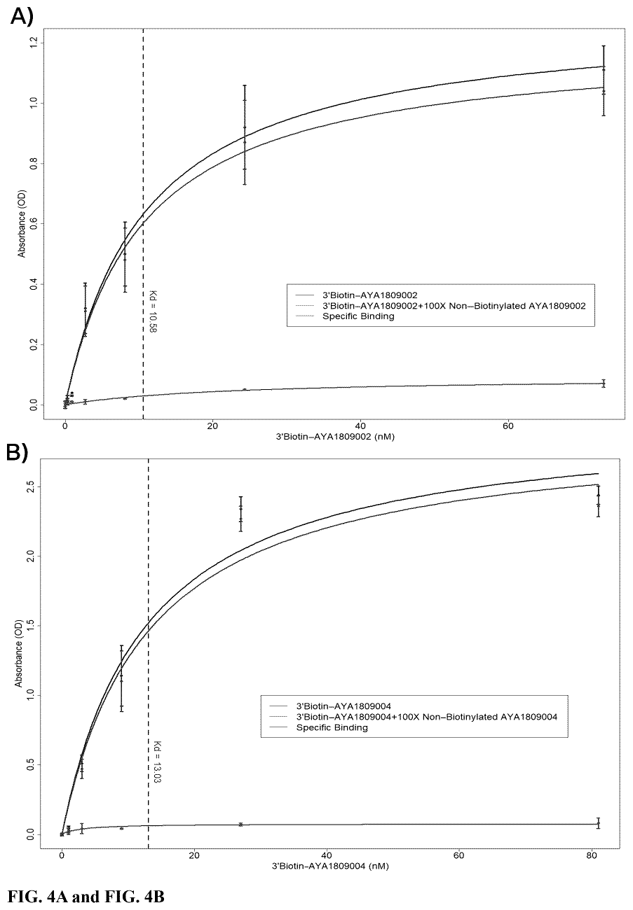

17. FIGS. 4A and 4B show ELISA-based competition assay to determine the affinity constant (Kd) for the aptamers AYA1809002 and AYA1809004. The indicated concentration of either 5′ Bioitin-AYA1809002 (4A) or 5′ Bioitin-AYA1809004 (4B) was incubated with thrombin protein immobilized on a 96-well ELISA plate in the absence or presence of a 100-fold excess of non-biotinylated AYA1809002 or AYA1809004, respectively. Absorbance was measured after incubation with streptavidin horseradish peroxidase bound to the biotinylated aptamer in the presence of the TMB substrate. Each bar shows the average of duplicate measurements. The binding affinity of AYA1809002 to thrombin is estimated to be 10 nM, while that of AYA1809004 is estimated to be 13 nM.

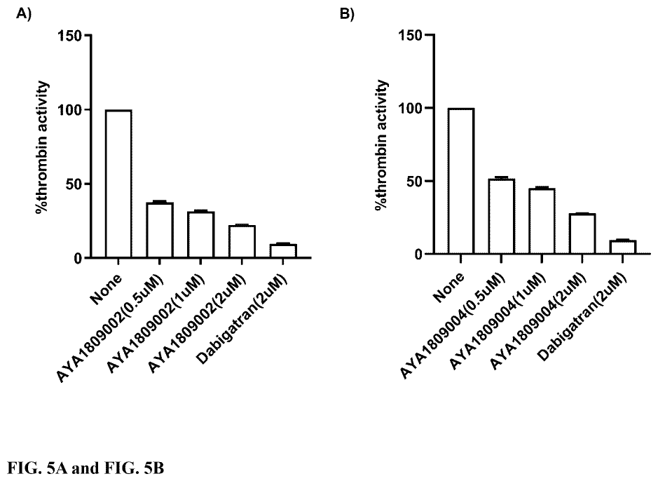

18. FIGS. 5A and 5B show Direct inhibition of thrombin activity by AYA1809002 (5A) and AYA1809004 (5B) in a dose-dependent manner. Different concentrations of aptamers, dabigatran at 2 uM, and buffer were added to their respective wells on the test plate. Subsequently, a thrombin enzyme mixture was added to each sample well. Following an incubation period of 15 min, a substrate mixture was introduced into each sample. Fluorescence measurements were taken using a SYNERGY/HTX multi-mode reader (BioTek) with excitation/emission wavelengths set at 360/460 nm. The measurements were recorded for 45 min at a temperature of 37° C. Each bar shows the average of duplicate measurements.

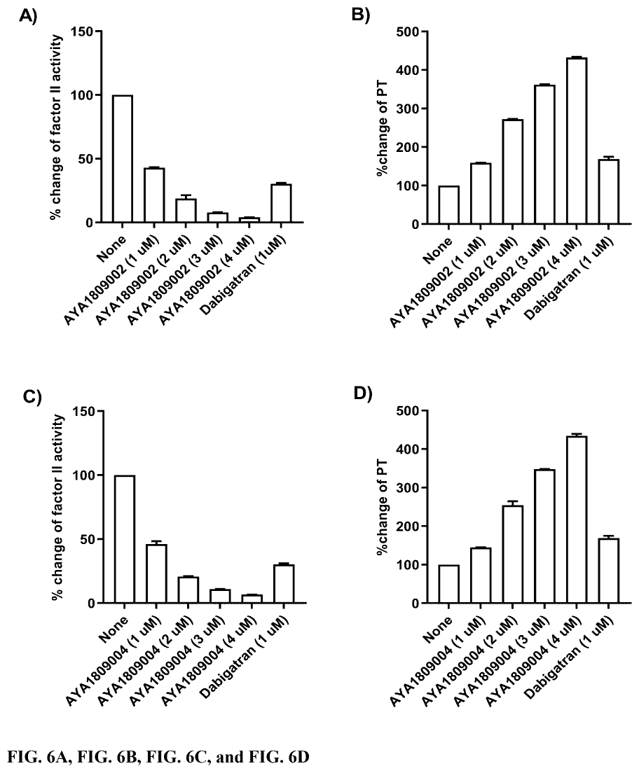

19. FIGS. 6A, 6B, 6C, and 6D show the effect of AYA1809002 and AYA1809004 aptamers on Factor II activity and Prothrombin time (PT) in whole blood in a dose-dependent manner. Whole human blood collected in citrate- treated tubes was incubated in the absence or presence of different concentrations of thrombin aptamers. Dabigatran at a concentration of 1 uM was used as a control. For measurement of Factor II activity and PT time on ACLTOP coagulation analyzer, plasma was separated by centrifugation. Inhibition of Factor II activity by the thrombin aptamers AYA1809002 (6A) and AYA1809004 (6C) was determined as compared to non-treated blood. Increase of PT by thrombin aptamers AYA1809002 (6B) and AYA1809004 (6D) was measured as compared to non-treated blood. Each bar shows the average of duplicate measurements.

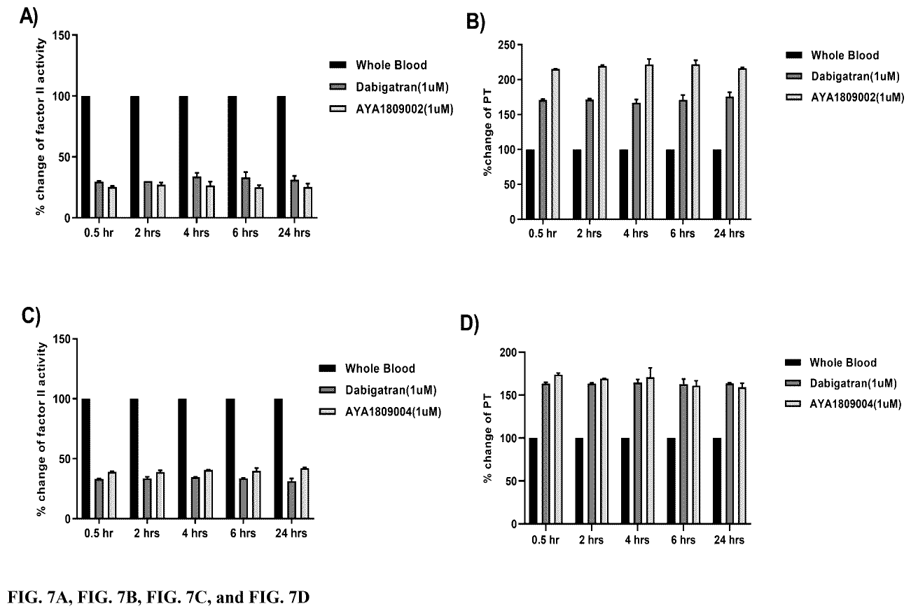

20. FIGS. 7A, 7B, 7C, and 7D show the stability of the selected aptamers in whole blood at room temperature. Citrated blood collected from a donor was incubated in the absence or presence of 1 uM of AYA1809002, AYA1809004, or Dabigatran for the indicated time. Factor II activity (7A and 7C) and PT time (7B and 7D) were measured for each sample. The relative activity was determined by dividing the measured activity of the treated whole blood sample by the activity of the corresponding untreated sample collected at the same time point. Each bar shows the average of duplicate measurements.

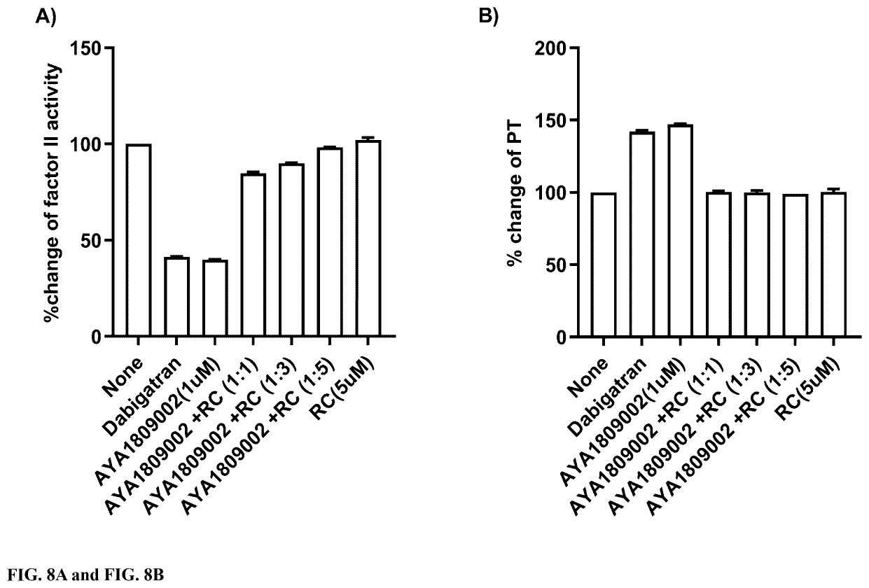

21. FIGS. 8A and 8B show restoration of Factor II activity and PT time using the reverse complement to AYA1809002 in whole blood. Citrated blood collected from a donor was incubated in the absence or presence of 1 mM AYA1809002 or dabigatran at room temperature. After 2 h, the indicated concentration of the reverse complement strand was added to the citrated blood sample. After an additional incubation period, plasma was collected per the existing protocol and Factor II activity (8A) and PT time (8B) were measured. Each bar shows the average of duplicate measurements.

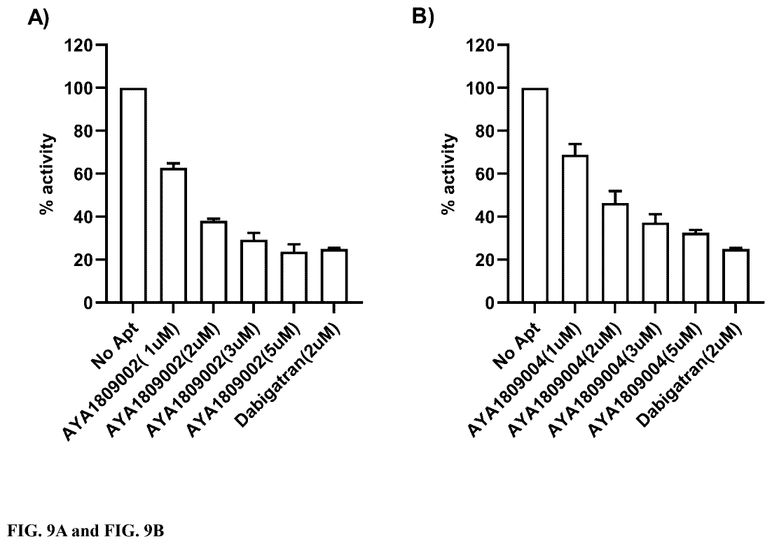

22. FIGS. 9A and 9b show Inhibition of clot-bound thrombin by the selected aptamers AYA1809002 (9A) and AYA1809004 (9B). Fibrin clots were formed from platelet-rich plasma. Different concentrations of aptamers, Dabigatran at 2 mM, and buffer were added to the respective wells containing the washed clots on the test plate. Following a 15 minute incubation period, a substrate mixture was introduced into each sample. Fluorescence measurements were taken using a SYNERGY/HTX multi-mode reader (BioTek) with the excitation/emission wavelengths set at 360/460 nm. The measurements were recorded for 90 min at a temperature of 37° C. Each bar shows the average of duplicate measurements.

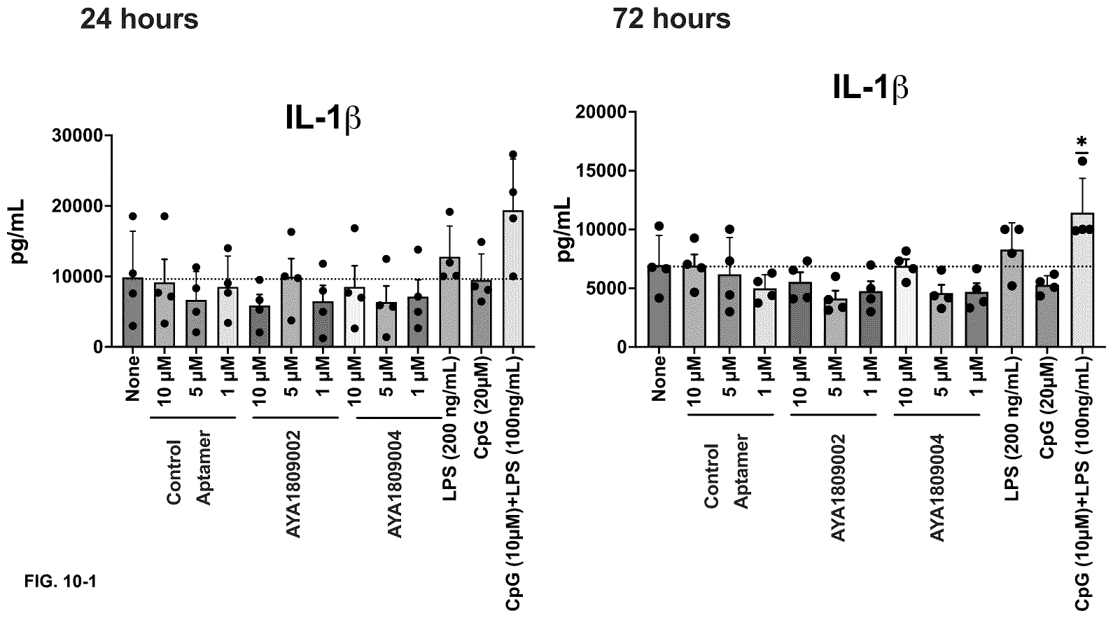

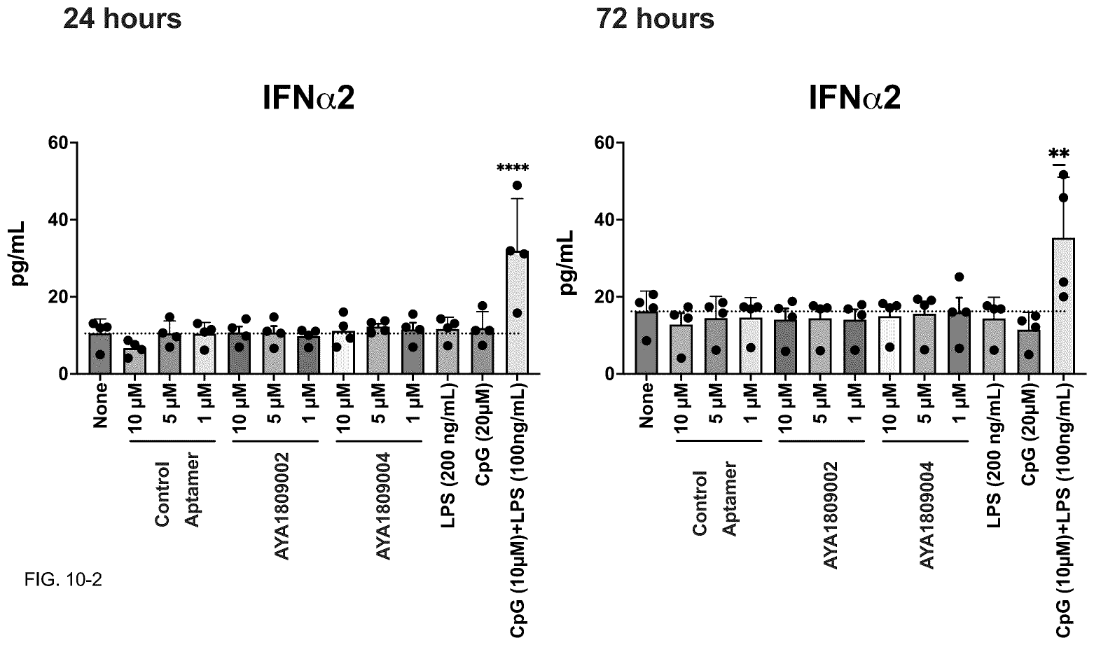

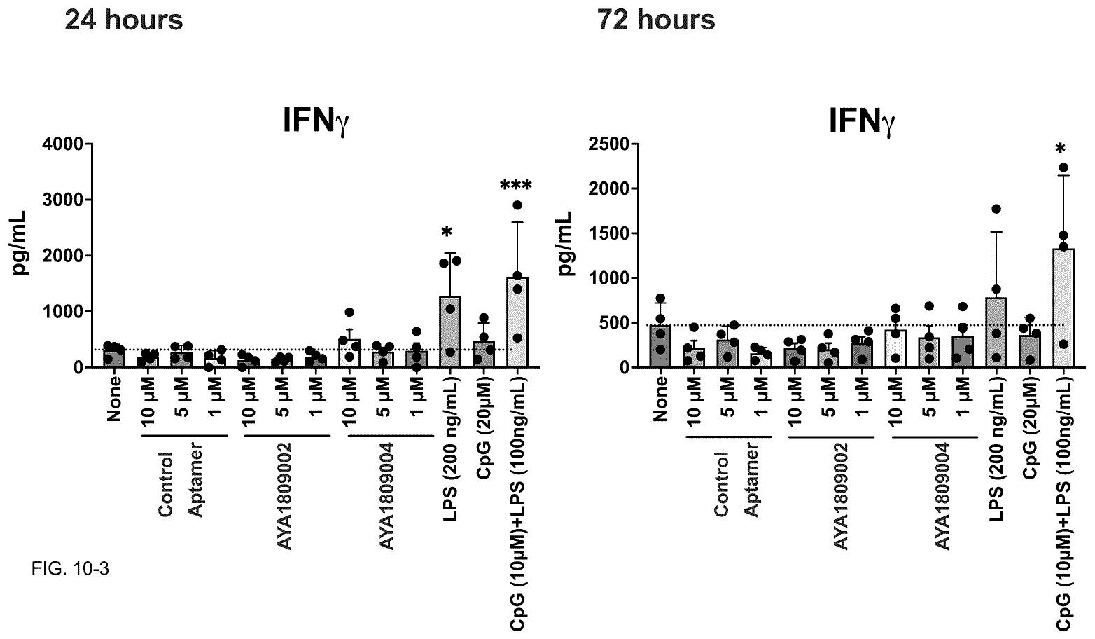

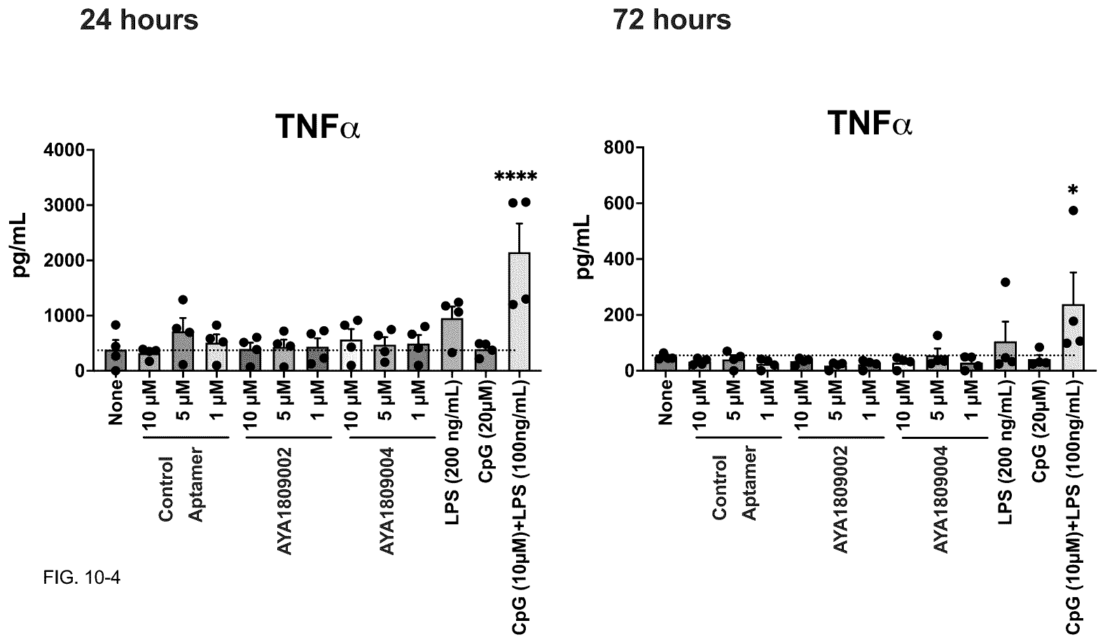

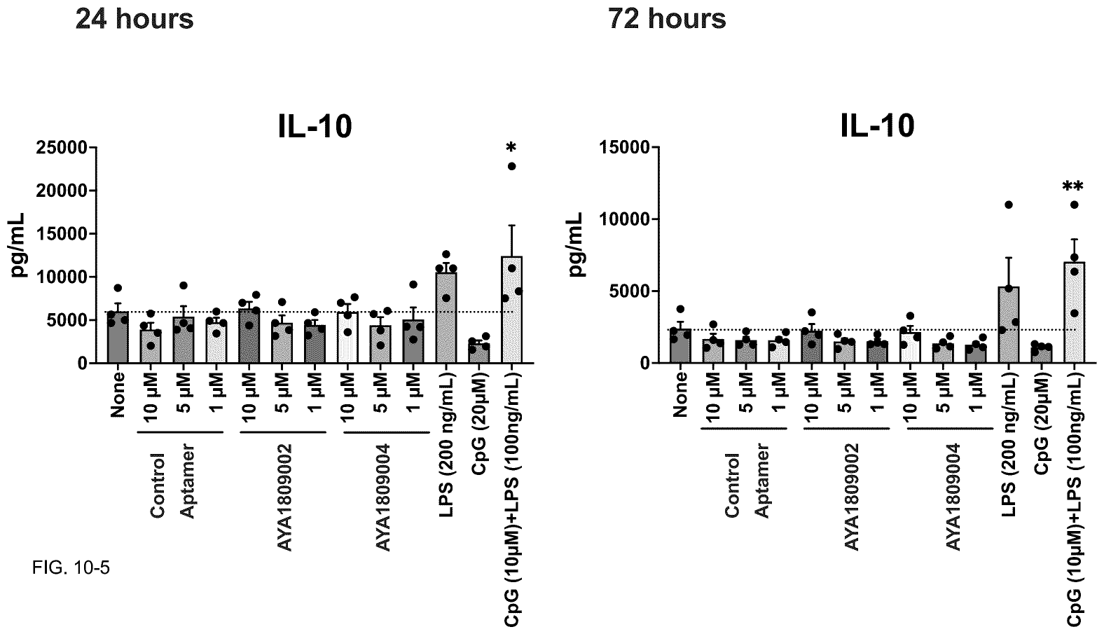

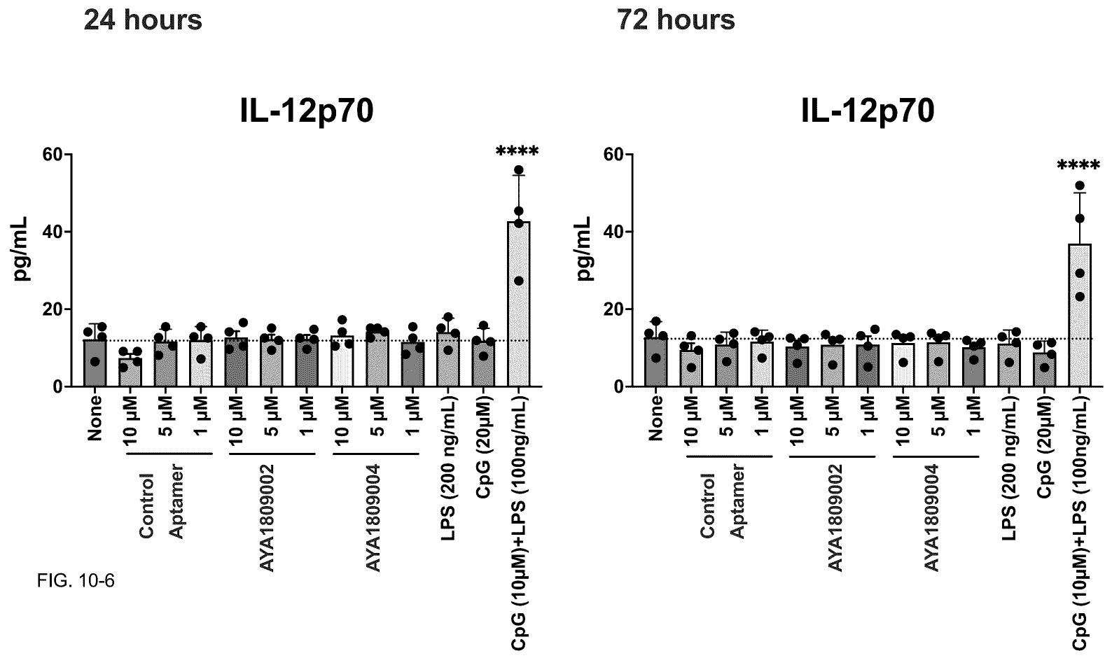

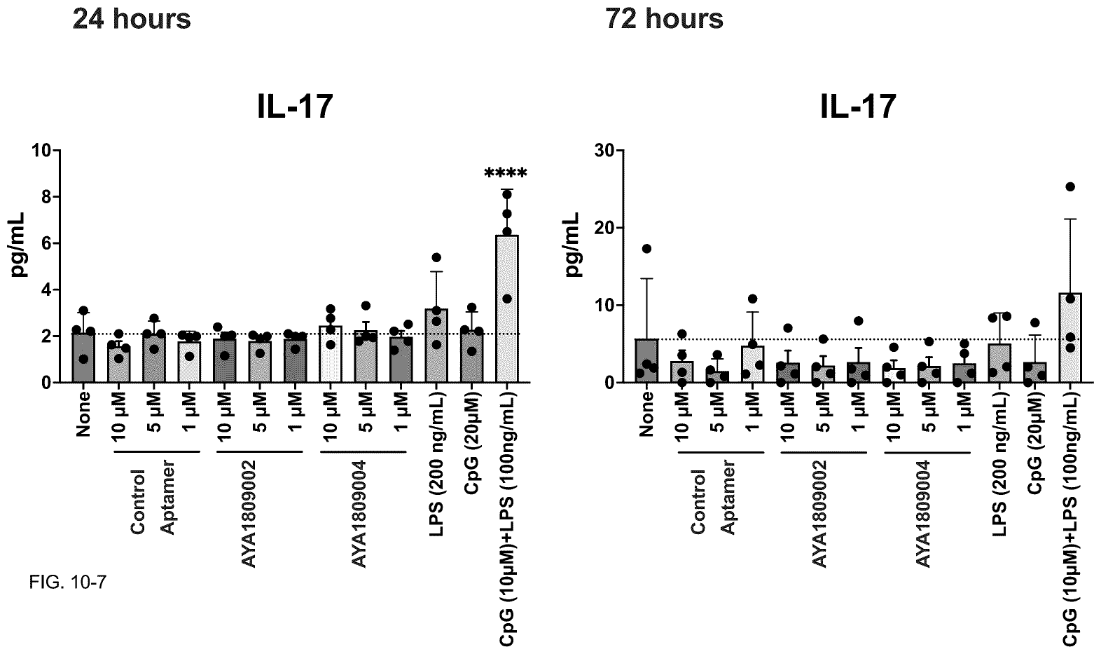

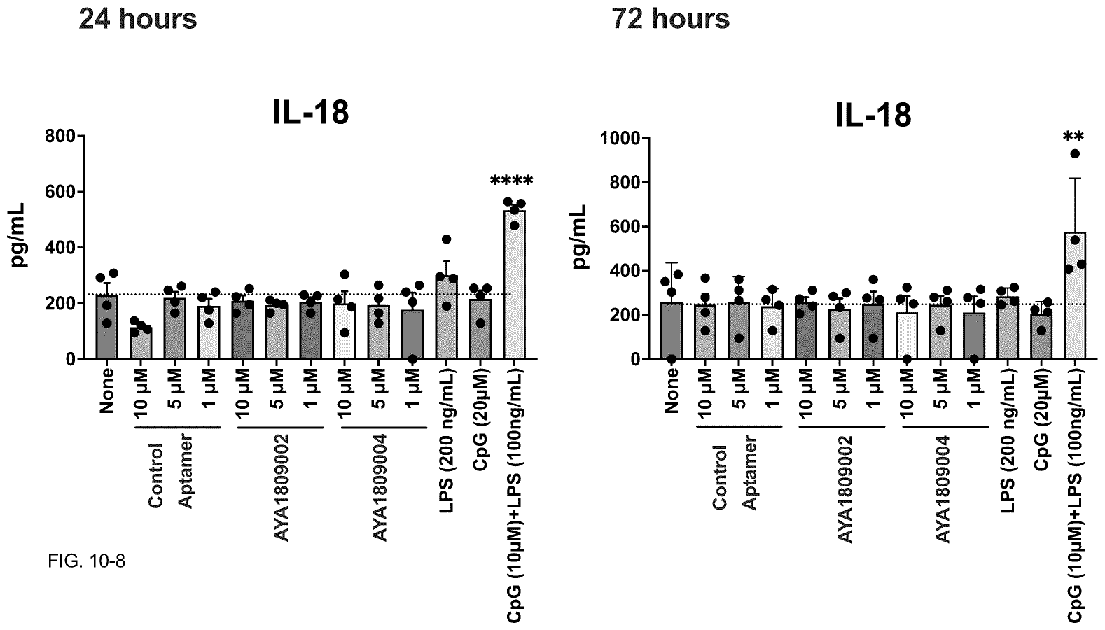

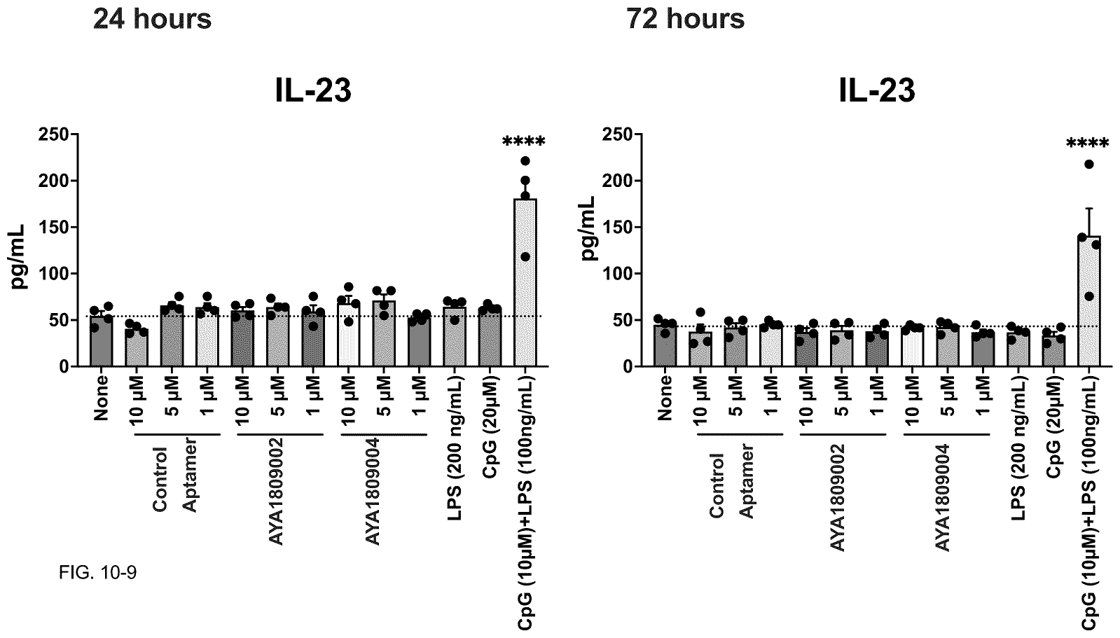

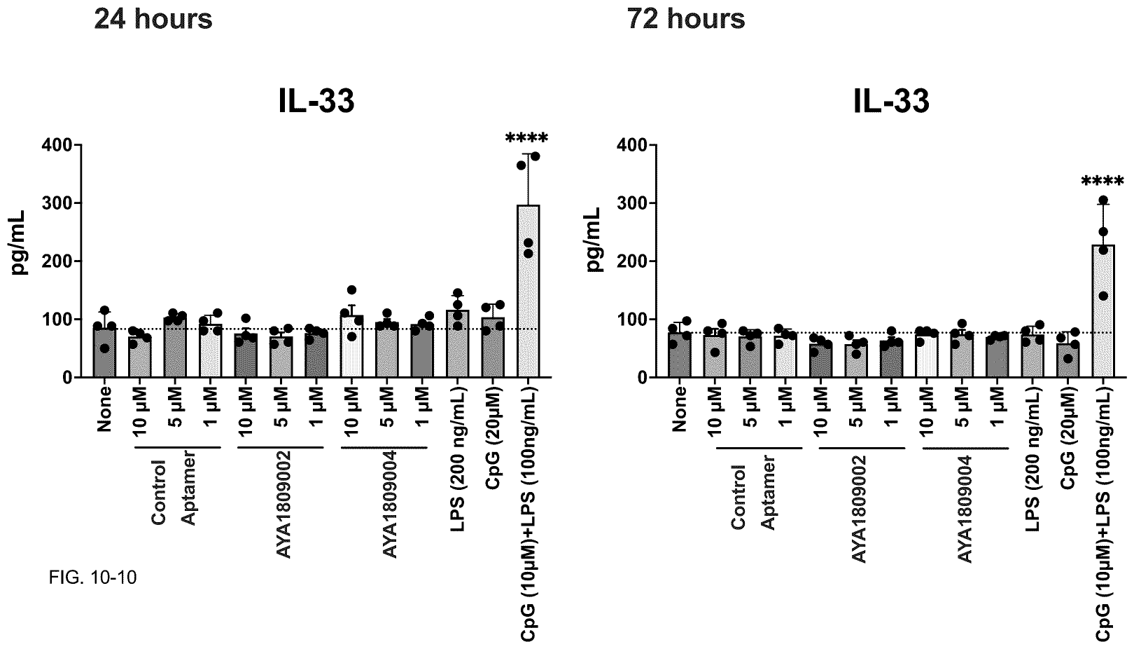

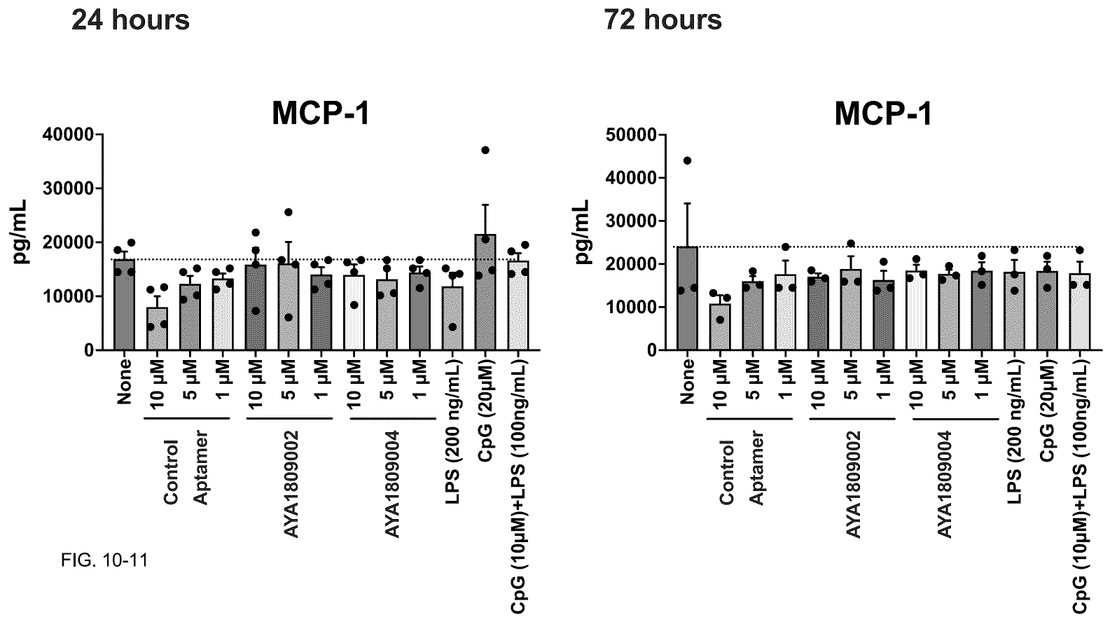

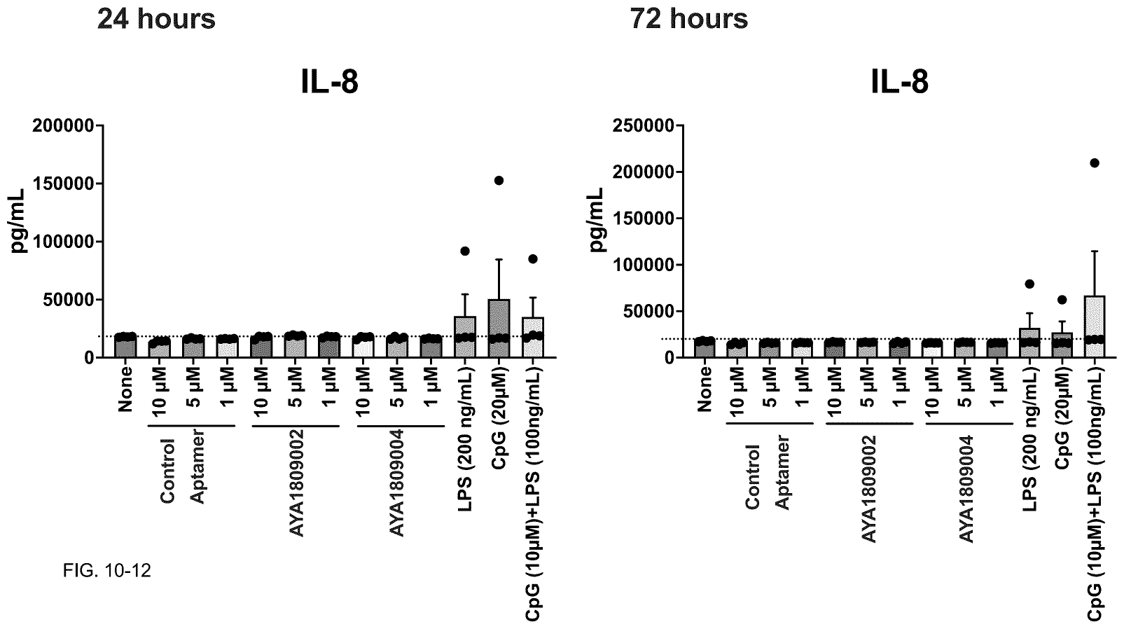

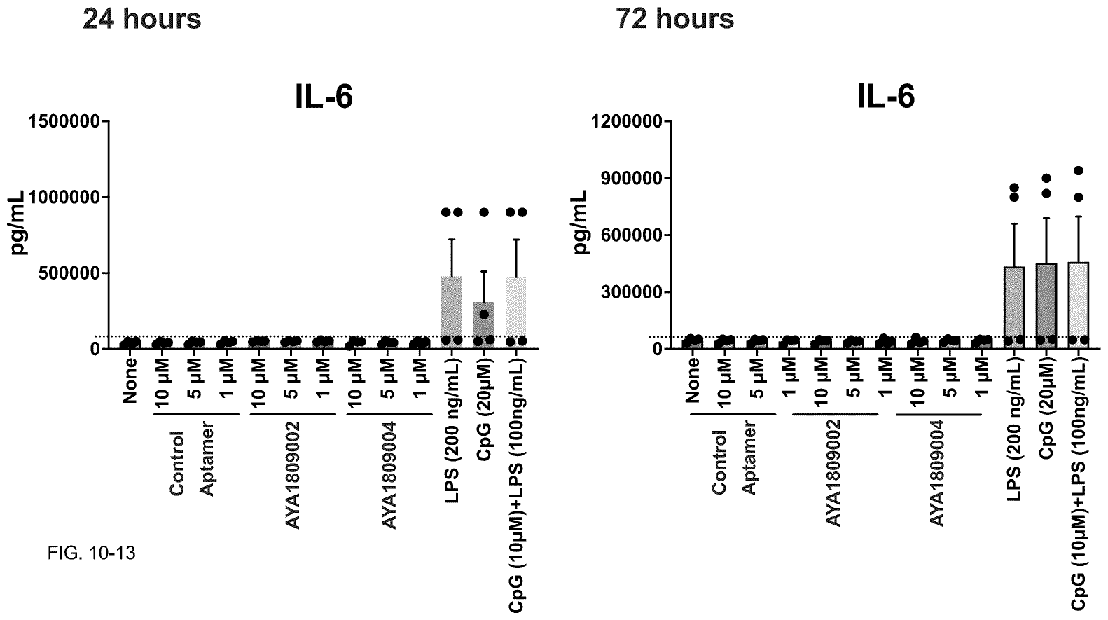

23. FIG. 10 shows Immunogenic response of AYA1809002 and AYA1809004. Human PBMCs were stimulated with or without AYA1809002, AYA1809004, control aptamer, LPS, ODN 1826, and LPS+ODN 1826 at 37° C. for 24 and 72 h. The amount of cytokines secreted from the hPBMCs was assessed using a LEGENDPLEX™ Human Inflammation Kit. Soluble analytes were quantified using flow cytometry and analyzed with BioLegend's LEGENDPLEX™ software. Each dot represents an individual donor (n=4). Data are graphed as the mean value±SEM. Thep values were determined with one-way ANOVA, Dunnett's multiple comparison test. * denotes p<0.05, ** denotes p<0.01, *** denotes p 50.001. **** denotes p<0.0001.

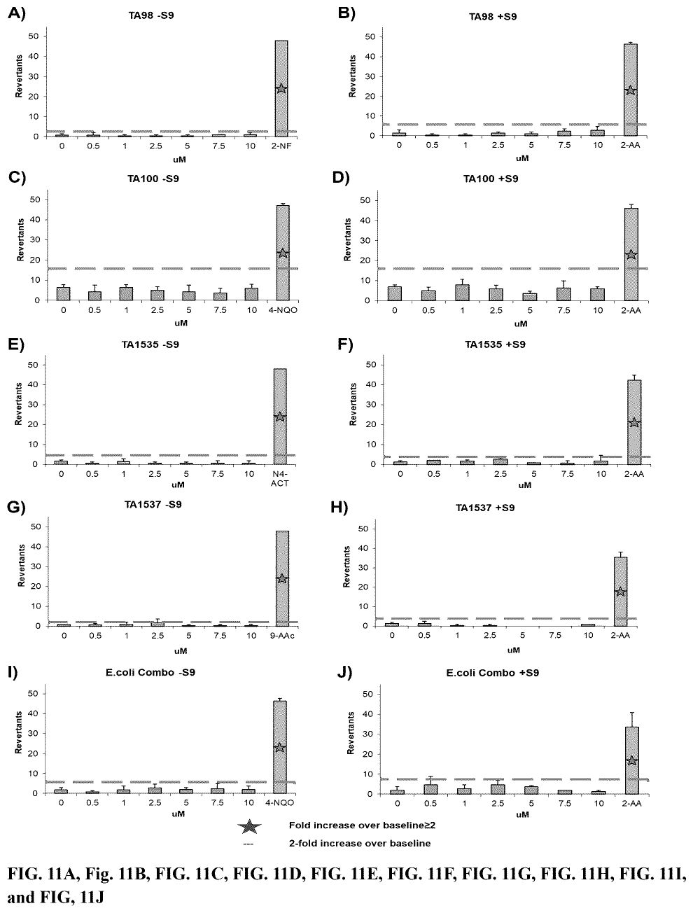

24. FIGS. 11A, 11B, 11C, 11D, 11E, 11F, 11G, 11H, 11I, and 11J show microbial mutagenicity test for AYA1809002. Assessment of the aptamer's mutagenic potential involved subjecting S. typhimurium strains (TA98, TA100, TA1535, TA1537) and E. coli strains (wp2[pkM101]and wp2 uvrA) to varied concentrations of the aptamer. Positive and negative controls were included. The aptamer AYA1809002 was applied at concentrations ranging from 0.5 mM to 10 mM. This assay was conducted both in the presence and absence of metabolic activation, which was facilitated by liver homogenate S9. Results were obtained from three independent experiments, and the data are presented as the mean±standard deviation (SD).

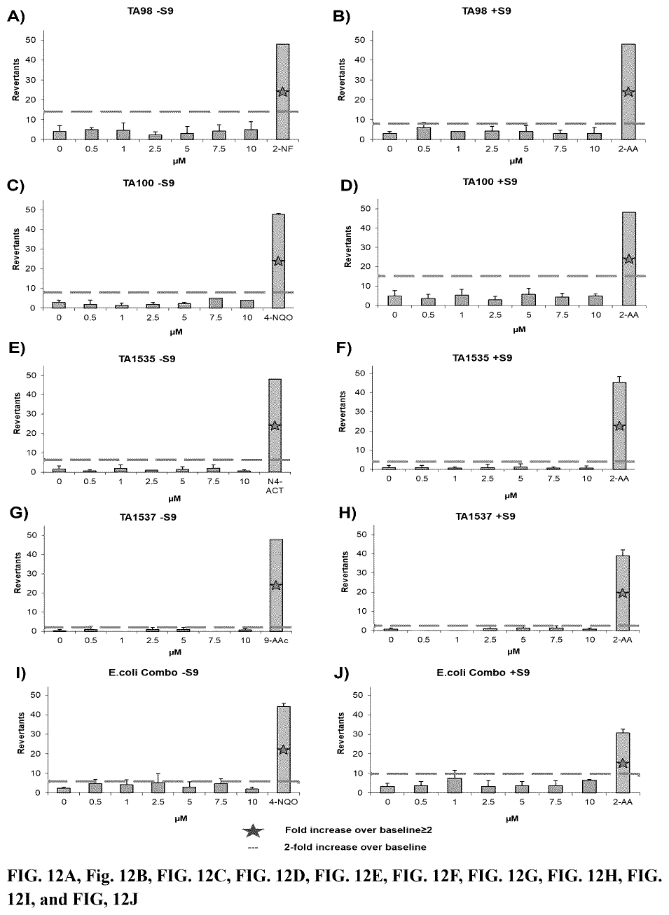

25. FIGS. 12A, 12B, 12C, 12D, 12E, 12F, 12G, 12H, 12I, and 12J show microbial mutagenicity test for AYA1809004. Assessment of the aptamer's mutagenic potential involved subjecting S. typhimurium strains (TA98, TA100, TA1535, TA1537) and E. coli strains (wp2[pkM101]and wp2 uvrA) to varied concentrations of the aptamer. Positive and negative controls were included. The aptamer AYA1809004 was applied at concentrations ranging from 0.5 mM to 10 mM. This assay was conducted in both the presence and absence of metabolic activation, which was facilitated by liver homogenate S9. Results were obtained from three independent experiments, and the data are presented as the mean±standard deviation (SD).

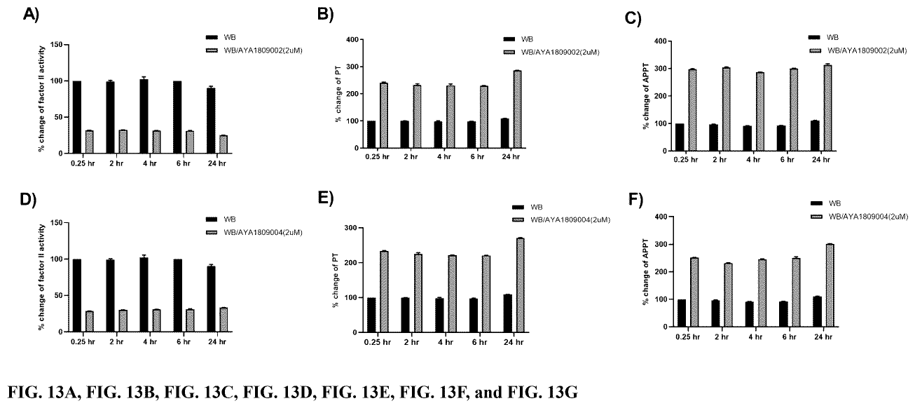

26. FIGS. 13A, 13B, 13C, 13D, 13E, 13F, and 13G show the stability of selected aptamers in whole blood at 37° C. Stability experiments were performed using whole blood at 37° C. Citrated blood collected from a donor was incubated in the absence or presence of 1 uM AYA1809002 or AYA1809004 at 37° C. Factor II activity (13A and 13D), PT time (13B and 13E) and APTT time (13C and 13F) were measured for each sample. The relative activity was determined by dividing the measured activity of the treated whole blood sample by the activity of the corresponding untreated sample collected at the same time point. Each bar is an average of duplicate measurements.

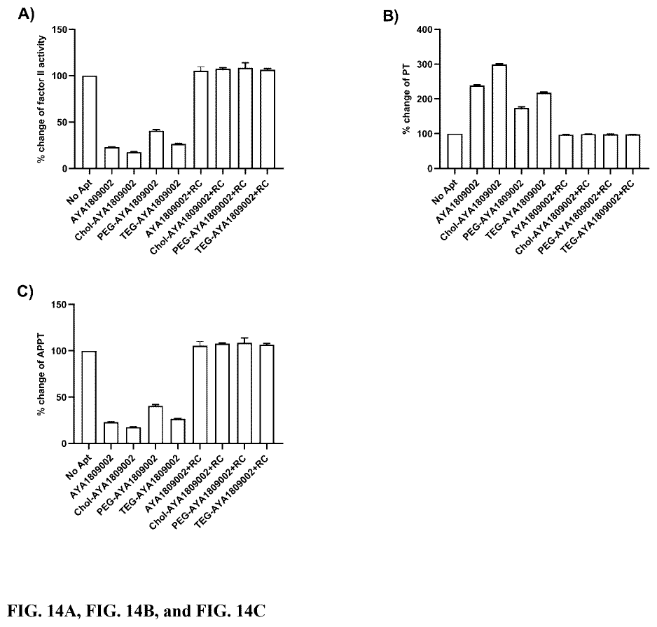

27. FIGS. 14A, 14B, and 14C show the effect of modified AYA1809002 aptamer on Factor II activity, PT, and APTT and reversal potential using Reverse Complement to AYA1809002. Human citrated plasma was incubated in the absence or presence of 2 uM of unmodified AYA1809002 or Cholesterol-, PEG- or TEG-modified AYA1809002 aptamers for 1 hour at room temperature. The indicated samples were supplemented with 5 μM of the reverse complement strand to AYA1809002. After an additional 2-hour incubation, Factor II activity (14A) and PT time (14B) and APTT time (14C) were measured. The relative activity of Factor II, PT and APTT time was determined by comparing the treated plasma sample to the untreated one. Each bar is an average of duplicate measurements.

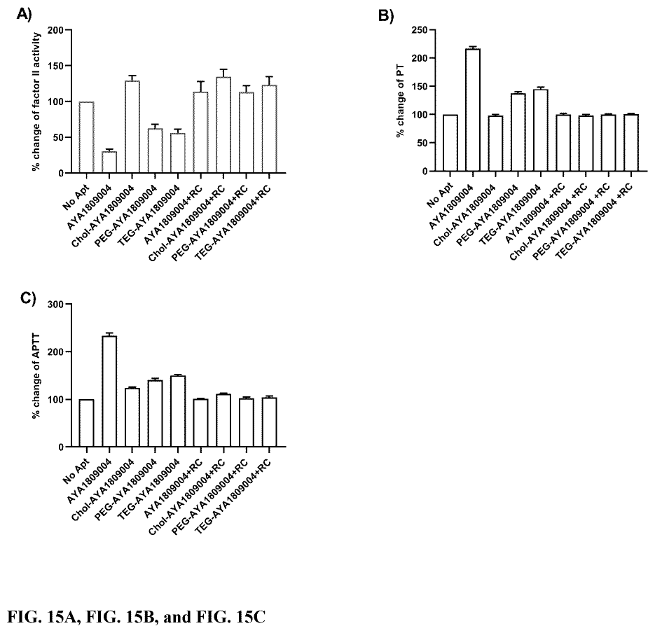

28. FIGS. 15A, 15B, and 15C show the effect of modified AYA1809004 aptamer on Factor II activity, PT, and APTT and reversal potential using Reverse Complement to AYA1809004. Human citrated plasma was incubated in the absence or presence of 2 mM of unmodified AYA1809004 or Cholesterol-, PEG- or TEG-modified AYA1809004 aptamers for 1 hour at room temperature. The indicated samples were supplemented with 5 mM of the reverse complement strand to AYA1809004. After an additional 2-hour incubation, Factor II activity (15A) and PT time (15B) and APTT time (15C) were measured. The relative activity of Factor II, PT and APTT time was determined by comparing the treated plasma sample to the untreated one. Each bar is an average duplicate measurements.

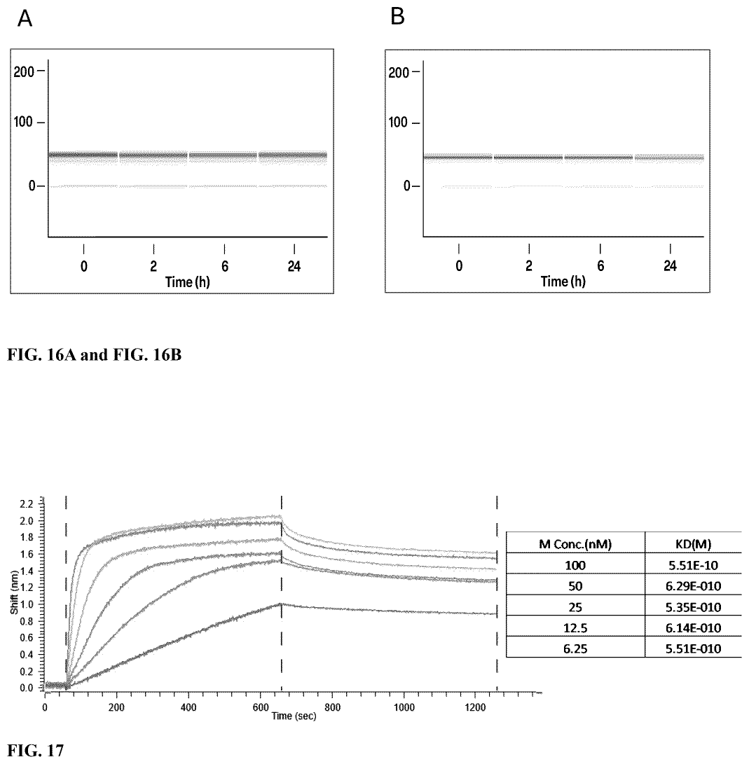

29. FIGS. 16A and 16B show the stability analysis of aptamers after 2-, 6- and 24-hours incubation in citrated blood at 37° C. . Analysis was performed using Fragment analyzer (Agilent Technologies). FIG. 16A shows aptamer AYA1809002 and FIG. 16B shows aptamer AYA1809004.

30. FIG. 17 shows the binding kinetics and affinity of the rat thrombin-aptamer interaction were measured label-free using BLI on GatorPrime. KD values are presented in the table. KD˜0.6 nM

### V. DETAILED DESCRIPTION

31. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

32. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

33. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10“as well as” greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

34. In this specification and in the claims which follow, reference will be made to a number of terms which shall be defined to have the following meanings:

35. “Optional” or “optionally” means that the subsequently described event or circumstance may or may not occur, and that the description includes instances where said event or circumstance occurs and instances where it does not.

36. An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

37. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

38. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

39. By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

40. By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

41. The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

42. The term “therapeutically effective” refers to the amount of the composition used is of sufficient quantity to ameliorate one or more causes or symptoms of a disease or disorder. Such amelioration only requires a reduction or alteration, not necessarily elimination.

43. The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

44. “Biocompatible” generally refers to a material and any metabolites or degradation products thereof that are generally non-toxic to the recipient and do not cause significant adverse effects to the subject.

45. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

46. A “control” is an alternative subject or sample used in an experiment for comparison purposes. A control can be “positive” or “negative.”

47. “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

48. A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

49. “Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

50. “Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

51. “Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

52. “Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

53. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

54. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular thrombin aptamer is disclosed and discussed and a number of modifications that can be made to a number of molecules including the thrombin aptamer are discussed, specifically contemplated is each and every combination and permutation of thrombin aptamer and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

55. In one aspect disclosed herein are isolated nucleic acids (aptamers) comprising the sequence as set forth in SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21, or any fragment or variant thereof comprising at least 90% sequence identity thereto.

56. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kUs from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of thrombin aptamers (such as, for example, AYA1809001, AYA1809002, AYA1809003, AYA1809004, AYA1809005, AYA1809006, AYA1809007, AYA1809008, and AYA1809009), the background protein could be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

57. Also disclosed herein is the RNA equivalent of any of the isolated nuclei acids (i.e., aptamers) disclosed herein.

58. In one aspect, disclosed herein are isolated nucleic acids (i.e., aptamers including, but not limited to SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21), wherein the nucleic acid (i.e., aptamer) further comprises a detectable tag (including, but not limited to a latex bead, magnetic bead, fluorescence label; fluorescent probe, chemiluminescent labels, radiolabels, and/or nanoparticle probe).

59. As used herein, a label or tag can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

60. Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4- Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4- I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs - AutoFluorescent Protein - (Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO- TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis- BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson -; Calcium Green; Calcium Green-1 Ca²⁺Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD- Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 1OGF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; ; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO- I PRO-3; Primuline; Procion Yellow; Propidium lodid (P1); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy- N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO- PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

61. A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

62. The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

63. Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

64. In one aspect, disclosed herein are isolated nucleic acids, wherein the nucleic acid (i.e., aptamer) further comprises a stabilizing moiety (including, but not limited to polyethylene glycol (PEG), triethylene glycol (TEG), or cholesterol) (such as, for example, SEQ ID NO: 16 (3′ PEG-AYA1809002), SEQ ID NO: 17 (3′ TEG-AYA1809002), SEQ ID NO: 18 (3′ Chol-AYA1809002), SEQ ID NO: 19 (3′ PEG-AYA1809004), SEQ ID NO: 20 (3′ TEG-AYA1809004), SEQ ID NO: 21 (3′ Chol-AYA1809004)).

65. Also disclosed herein are aptamer antidotes comprising the sequence the reverse complement of aptamers disclosed herein (such as, for example, SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21). In one aspect, the aptamer antidote comprises the sequence as set forth in SEQ ID NO: 23 or SEQ ID NO: 24.

1. Nucleic acids

66. There are a variety of molecules disclosed herein that are nucleic acid based, including for example the nucleic acids that encode, for example, the thrombin aptamers AYA1809001 (SEQ ID NO: 1), AYA1809002 (SEQ ID NO: 2), AYA1809003 (SEQ ID NO: 3), AYA1809004 (SEQ ID NO: 4), AYA1809005 (SEQ ID NO: 5), AYA1809006 (SEQ ID NO: 6), AYA1809007 (SEQ ID NO: 7), AYA1809008 (SEQ ID NO: 8), and AYA1809009 (SEQ ID NO: 9) and/or the aptamer antidotes set forth in SEQ ID NO: 23 and SEQ ID NO: 24. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### A) Nucleotides and Related Molecules

67. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

68. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′—O-methoxyethyl, to achieve unique properties such as increased duplex stability.

69. Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl—O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙ CH₃, —O(CH₂)ₙ—ONH₂, and —O(CH₂)ₙON[(CH₂)ₙ CH₃)]2, where n and m are from 1 to about 10. 70. Other modifications at the 2′ position include but are not hinted to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar.

71. Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

72. It is understood that nucleotide analogs need only contain a single modification, but may also contain multiple modifications within one of the moieties or between different moieties.

73. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

74. Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

75. It is also understood in a nucleotide substitute that both the sugar and the phosphate moieties of the nucleotide can be replaced, by for example an amide type linkage (aminoethylglycine) (PNA).

76. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

77. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

78. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

### 2. Homology/Identity

79. It is understood that one way to define any known variants and derivatives or those that might arise, of the disclosed genes and proteins herein is through defining the variants and derivatives in terms of homology to specific known sequences. For example, SEQ ID Nos: 1, 2, 3, 4, 5, 67, 8, and 9 set forth a particular sequence of thrombin aptamers, namely AYA1809001, AYA1809002, AYA1809003, AYA1809004, AYA1809005, AYA1809006, AYA1809007, AYA1809008, and AYA1809009. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

80. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J MoL Biol. 48: 443 (1970), by the search for similarity method of Pearson and Lipman, Proc. Nat. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

81. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 3. Delivery of the Compositions to Cells

82. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### A) Nucleic Acid-Based Delivery Systems

83. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

84. As used herein, plasmid or viral vectors are agents that transport the disclosed aptamers, such as SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21 and aptamer antidotes, such as SEQ ID NO: 23 and SEQ ID NO: 24 into the cell without degradation and include a promoter yielding expression of the gene in the cells into which it is delivered. In some embodiments the aptamers and/or aptamer antidoes are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

85. Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

86. A retrovirus is an animal virus belonging to the virus family of Retroviridae, including any types, subfamilies, genus, or tropisms. Retroviral vectors, in general, are described by Verma, I. M., Retroviral vectors for gene transfer.

87. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

88. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

89. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J Clin. Invest. 92:1580-1586 (1993); Kirshenbaum, J Clin. Invest. 92:381-387 (1993); Roessler, J Clin. Invest. 92:1085-1092 (1993); Moullier, Nature Genetics 4:154-159 (1993); La Salle, Science 259:988-990 (1993); Gomez-Foix, J Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J Virology 65:6061-6070 (1991); Wickham et al., Cell 73:309-319 (1993)). 90. A viral vector can be one based on an adenovirus which has had the El gene removed and these virons are generated in a cell line such as the human 293 cell line. In another preferred embodiment both the El and E3 genes are removed from the adenovirus genome.

### (3) Adeno-Associated Viral Vectors

91. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

92. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

93. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

94. The disclosed vectors thus provide DNA molecules which are capable of integration into a mammalian chromosome without substantial toxicity.

95. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

96. Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson,. Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes. 97. Other useful systems include, for example, replicating and host-restricted non-replicating vaccinia virus vectors.

### B) Non-Nucleic Acid Based Systems

98. The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

99. Thus, the compositions can comprise, in addition to the disclosed aptamers, such as SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21 and aptamer antidotes, such as SEQ ID NO: 23 and SEQ ID NO: 24, vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

100. In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

101. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

102. Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

103. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### C) In Vivo/Ex Vivo

104. As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

105. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 4. Pharmaceutical Carriers/Delivery of Pharmaceutical Products

106. In one aspect, the isolated nucleic acid (i.e., aptamers) disclosed herein (including, but not limited to SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21) and/or aptamer antidotes disclosed herein (including, but not limited to SEQ ID NO: 23 and SEQ ID NO: 24) can be in a pharmaceutical composition (such as, for example, an active component in a pharmaceutical composition). As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

107. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

108. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

109. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

110. The compositions, including antibodies, can be used therapeutically in combination with a pharmaceutically acceptable carrier.

111. Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

112. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

113. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

114. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

115. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

116. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

117. Compositions for oral administration include powders or granules, suspensions or solutions in water or non-aqueous media, capsules, sachets, or tablets. Thickeners, flavorings, diluents, emulsifiers, dispersing aids or binders may be desirable..

118. Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

119. Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### 5. Kits

120. Disclosed herein are kits that are drawn to reagents that can be used in practicing the methods disclosed herein. In one aspect, disclosed herein are kits comprising any of the isolated nucleic acids (i.e., aptamers) disclosed herein. For example, disclosed herein are kits comprising one or more of SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, and/or SEQ ID NO: 21. In some aspects, the kit may further comprises an aptamer antidote (such as, for example, SEQ ID NO: 23 and/or SEQ ID NO: 24). Thus, in one aspect, disclosed herein are kits wherein the isolated nucleic acid (i.e., the aptamer) comprises SEQ ID NO: 2, SEQ ID NO: 16, SEQ ID NO: 17, and/or SEQ ID NO: 18 and the aptamer antidote comprises SEQ ID NO: 23 or wherein the isolated nucleic acid (i.e., aptamer) comprises SEQ ID NO: 4, SEQ ID NO: 19, SEQ ID NO: 20, and/or SEQ ID NO: 21 and the aptamer antidote comprises SEQ ID NO: 24.

121. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for detecting the presence of a viral infection (such as, for example, a coronaviral infection) or simply an S protein of a coronavirus comprising any nucleotide encoding the amino acids set forth in SEQ ID Nos: 1-158, as well as any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.

122. While various embodiments of the present disclosure have been described above, it should be understood that they have been presented by way of example only, and not of limitation. Likewise, the various diagrams may depict an example architectural or other configuration for the invention, which is provided to aid in understanding the features and functionality that can be included in the invention. The invention is not restricted to the illustrated example architectures or configurations, but the desired features can be implemented using a variety of alternative architectures and configurations.

123. Indeed, it will be apparent to one of skill in the art how alternative functional configurations can be implemented to implement the desired features of the present disclosure. Additionally, with regard to flow diagrams, operational descriptions and method claims, the order in which the steps are presented herein shall not mandate that various embodiments be implemented to perform the recited functionality in the same order unless the context dictates otherwise.

124. Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present invention should not be limited by any of the above-described exemplary embodiments.

### C. Methods of Treating Thrombosis

125. It is understood and herein contemplated that the disclosed aptamers are designed specifically to treat thrombosis, thrombotic conditions, and/or any inflammatory condition where the inflammation affects the vasculature (including, but not limited to such as thromboembolic stroke, ischemia, heart attack, arterial thrombosis, deep vein thrombosis (DVT), disseminated intravascular coagulation (DIC), venous thromboembolism (VTE), central venous sinus thrombosis, cavernous sinus thrombosis, central retinal vein occlusion, branch retinal vein occlusion, Paget-Schroetter disease, Budd-Chiari syndrome, splanchnic venous thrombosis, renal vein thrombosis, ovarian vein thrombosis, jugular vein thrombosis, or pulmonary embolism (PE)) comprising administering to a subject an anticoagulant (such as, for example bivalirudin (ANGIOMAX®), antithrombin III, argatroban (ACOVA®), dabigatran (PRADAXA®), heparin, warfarin (COUMADIN®), apixaban (ELIQUIS®), edoxaban (SAVAYSA®), enoxaparin (LOVENOX®), fondaparinux (ARIXTRA®), and rivaroaxaban (XARELTO®)), or the symptoms of these conditions. Accordingly, in one aspect, disclosed herein are methods of treating, inhibiting, decreasing, reducing, and/or ameliorating thrombosis, a thrombotic condition, or and/or any inflammatory condition where the inflammation affects the vasculature comprising administering to a subject an anticoagulant (such as, for example bivalirudin (ANGIOMAX®), antithrombin III, argatroban (ACOVA®), dabigatran (PRADAXA®), heparin, warfarin (COUMADIN®), apixaban (ELIQUIS®), edoxaban (SAVAYSA®), enoxaparin (LOVENOX®), fondaparinux (ARIXTRA®), and rivaroaxaban (XARELTO®)) (including, but not limited to such as thromboembolic stroke, heart attack, deep vein thrombosis (DVT), venous thromboembolism (VTE), disseminated intravascular coagulation (DIC), or pulmonary embolism (PE)), or the symptoms thereof in a subject in need thereof comprising administering to the subject one or more of the isolated nucleic acids (i.e., aptamers) disclosed herein. For example, disclosed herein are methods of treating, inhibiting, decreasing, reducing, and/or ameliorating thrombosis or a thrombotic condition, and/or any inflammatory condition where the inflammation affects the vasculature comprising administering to a subject an anticoagulant (such as, for example bivalirudin (ANGIOMAX®), antithrombin III, argatroban (ACOVA®), dabigatran (PRADAXA®), heparin, warfarin (COUMADIN®), apixaban (ELIQUIS®), edoxaban (SAVAYSA®), enoxaparin (LOVENOX®), fondaparinux (ARIXTRA®), and rivaroaxaban (XARELTO®)) or the symptoms thereof in a subject in need thereof comprising administering to the subject one or more isolated nucleic acids (aptamers) comprising the sequence as set forth in SEQ ID NO: 1 (AYA1809001), SEQ ID NO: 2 (AYA1809002), SEQ ID NO: 3 (AYA1809003), SEQ ID NO: 4 (AYA1809004), SEQ ID NO: 5 (AYA1809005), SEQ ID NO: 6 (AYA1809006), SEQ ID NO: 7 (AYA1809007), SEQ ID NO: 16 (3′ PEG-AYA1809002), SEQ ID NO: 17 (3′ TEG-AYA1809002), SEQ ID NO: 18 (3′ Chol-AYA1809002), SEQ ID NO: 19 (3′ PEG-AYA1809004), SEQ ID NO: 20 (3′ TEG-AYA1809004), SEQ ID NO: 21 (3′ Chol-AYA1809004).

126. In some aspects, it is understood and herein contemplated that treatment with any of the disclosed aptamers can cause adverse effects associated with the aptamer treatment. Thus, it is advantageous for the treating physician to be able to cease or reduce the effects of the aptamer. Accordingly, disclosed herein are methods of treating, inhibiting, decreasing, reducing, and/or ameliorating an adverse event from administration of an aptamer therapy comprising administering to a subject having previously received the aptamer therapy, any of the aptamer antidotes disclosed herein (such as, for example SEQ ID NO: 23 or SEQ ID NO: 24). Thus, disclosed herein are methods of treating, inhibiting, decreasing, reducing, and/or ameliorating an adverse event from administration of an aptamer therapy wherein the aptamer comprises SEQ ID NO: 2, SEQ ID NO: 16, SEQ ID NO: 17, and/or SEQ ID NO: 18 and the aptamer antidote comprises SEQ ID NO: 23; or wherein the aptamer comprises SEQ ID NO: 4, SEQ ID NO: 19, SEQ ID NO: 20, and/or SEQ ID NO: 21 and the aptamer antidote comprises SEQ ID NO: 24.

### D. Examples

127. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: High-Affinity Thrombin Aptamers for Advancing Coagulation Therapy: Balancing Thrombin Inhibition for Clot Prevention and Effective Bleeding Management with Antidote

128. Aptamers are RNA and DNA oligonucleotides that bind to specific target molecules with high affinity and specificity, in which they are similar to antibodies; however, they have many advantages over antibodies, such as long shelf life, ease of modification, cost-effectiveness, and lower immunogenicity. In the coagulation cascade, aptamers have been developed to interact with multiple coagulation factors and cofactors, offering antidote-mediated controllability that safeguards the intrinsic coagulation capacity from exhaustion. As a result, aptamers have recently drawn a great deal of attention for their potential to regulate the activity of thrombin.

129. The first anti-thrombin DNA aptamer (HDT, 5′-GGTTGGTGTGGTTGG-3′) (SEQ ID NO: 10) was synthesized by Dr. Toole in 1992; it is a 15-mer aptamer that forms a stable G-quadruplex binding to exosite I on prothrombin and thrombin. HD1 demonstrates a comprehensive inhibitory effect by targeting multiple pathways, including fibrinogen and FV cleavage, prothrombinase inhibition, and blocking platelet PARI interaction with exosite I. This multifaceted action results in the effective suppression of thrombin-mediated platelet activation and aggregation. In vivo studies have shown that HD1 inhibits>80% of clot-bound thrombin, as compared to 35% with heparin. Due to its short half-life and rapid clearance, HD1 needs to be administered by infusion in large amounts, which poses a challenge in the dosing and monitoring of the drug. For these reasons, HD1 was ruled out for phase I clinical trials. Additional anti-thrombin aptamers that bind to exosite I of thrombin were identified later, such as NU172, RA36, R9D-14T, and RE31. Among these, NU172 is the only aptamer that entered phase II clinical trials (NCT00808964); at present, there is a lack of available information on its current status. HD22 (29-mer, 5′-AGTCCGTGGTAGGGCAGGTTGGGGTGACT-3′) (SEQ ID NO: 11)is another well-known aptamer. This aptamer recognizes exosite II of thrombin, a crucial region for activation of factors V and VIII, as well as for the mediation of heparin binding. As a result, HD22 inhibits the activation of factors V and VIII rather than that of fibrinogen. Toggle-25t, an RNA aptamer, is another aptamer that binds to exosite II of thrombin. 130. To enhance the inhibitory potency of aptamers, studies have targeted multiple sites of thrombin for inhibition. As a result, several anti-thrombin bivalent aptamers have been designed by chemically linking an aptamer that binds to exosite I with another aptamer that targets exosite II. Examples of such aptamers include HD1-22, a bivalent aptamer that connects HD1 and HD22(Kd=0.65 nM), as well as RNV220 and RNV220-T, both bivalent aptamers that connect HD1 and HD22 by TEG or poly-dT linkage. Recently, another bivalent aptamer linking M08s-1 and TBA29 was developed for heparin-induced thrombocytopenia, showing significantly stronger anticoagulant activity than NU172. While numerous anti-thrombin aptamers have been designed and developed, no anti- thrombin aptamer has reached advanced stages in clinical trials or received approved from the FDA. One major challenge in their clinical translation pertains to their susceptibility to nuclease degradation and compromised pharmacokinetic properties, requiring higher dosing regiments. Consequently, the development of new thrombin aptamers remains crucial as a means to broaden the range of therapeutic options, enhance treatment effective- ness, overcome existing limitations, and propel the field towards more personalized and effective approaches in the management of thrombotic disorders. 131. In this study, we employed systematic evolution of ligands by exponential enrichment (SELEX) to select aptamers that bind to the active site of thrombin. Several anti-thrombin aptamers with binding high affinity were successfully generated. The two most potent aptamers, AYA1809002 and AYA1809004, exhibit high affinity for their target, with affinity constants (Kd) of 10 nM and 13 nM, respectively. Furthermore, they demonstrate in vitro assay activity by effectively decreasing Factor II activity and increasing both prothrombin time (PT) and activated partial thromboplastin time (APTT) in plasma and whole blood in a dose-dependent manner. Crucially, the thrombin inhibitory activity of these aptamers can be effectively reversed through the use of reverse complement sequences. This feature provides a valuable mechanism to counteract their anticoagulant effects, enabling timely and precise intervention in emergency scenarios. Our results show that these two aptamers are stable in whole blood at room temperature and 37° C. for at least 24 h. To enhance their stability in the bloodstream, both aptamers were chemically modified through crosslinking with TAG, PEG, or cholesterol moieties (Table 2).

These modified aptamers successfully demonstrated in vitro activity, indicating that they retain their functionality. Additionally, both of these thrombin aptamers were determined to be safe, without any observed immunogenic or mutagenic effects. The findings indicate that AYA1809002 and AYA1809004 are effective and safe anti-thrombin candidate aptamers that can be used to treat thrombosis.

### a) Materials and Methods

### (1) Immobilization of Thrombin Protein on Magnetic Beads

132. The immobilization of human alpha-thrombin native protein (ThermoFisher Scientific, cat #RP-43100, Waltham, MA, USA) on Pierce™ NHS-Activated Magnetic Beads (ThermoFisher Scientific, cat #88827, Waltham, MA, USA) was performed as described by standard protocol, as follows. One mg of thrombin (MW 36.7 kDa) was dissolved in coupling buffer (0.1 M NaHCO₃, pH 8.3 containing 0.15 M NaCl) to a concentration of 0.7 mg/mL and dialyzed against 500 mL coupling buffer overnight. One ml of magnetic beads (10 mg of beads) was placed on the magnetic stand, the supernatant was discarded, and the beads were resuspended in 3 mL of ice-cold 1 mM hydrochloric acid, vortexed for 15 s, and placed on the magnetic stand to collect the beads; the supernatant was then discarded. Coupling solution containing thrombin was added to the functionalized beads and the mixture was rotated for 2 h at room temperature. The excess of ligand was washed out with coupling buffer followed by 0.1 M glycine buffer, pH 2.5. Remaining active groups were blocked using 3 M ethanolamine, pH 9.0. After 2 h, the incubation beads were washed and resuspended in selection buffer (20 mM Tris pH 7.5, 150 mM NaCl). Binding of thrombin was quantitated and confirmed using Piece BCA protein Assay kit (ThermoFisher Scientific, cat #23227, Waltham, MA, USA). The final concentration was estimated as 36 μg of protein per mg beads, or 1 nmol of protein per mg of beads. The same procedure was performed with the beads without thrombin protein in coupling buffer for negative selection.

### (2) SELEX

133. The systematic evolution of ligands by exponential enrichment (SELEX) procedure was performed. A synthetic single stranded DNA library consisted of a random sequence of 40 nucleotides flanked by two primers binding se- quences 5′ TAGGGAAGAGAAGGACATATGAT(N40)TTGACTAGTACATGACCACTTGA 3′ (SEQ ID NO: 12) (TriLink biotechnologies, cat #O-32140, 9955 Mesa Rim Road, San Diego, CA, 92121, USA), and 5′-Biotin Reverse primer was used for single strand separation. Throughout the subsequent SELEX rounds, the initial ssDNA library (10 nmol) was dissolved in 100 μL of DNase and RNase free water plus 100 μL of selection buffer (20 mM Tris pH 7.5, 150 mM NaCl). The diluted library was denatured using Hybex incubator at 95° C. for 10 min and cooled slowly on ice for 10 min then at room temperature for 30 min to allow the formation of stable secondary structures. Selection buffer was added up to 1 mL total volume of the library. This library was incubated with 300 μL (3 mg) of the magnetic beads (no thrombin) equilibrated with selection buffer for 2 h at room temperature (negative selection). The beads were collected using the magnetic stand and the supernatant was applied to 1 mg of the thrombin beads equilibrated with selection buffer. After 2 h incubation at room temperature and washing in the selection buffer, the bound ssDNA pool was eluted under alkaline conditions (200 μL of 0.15 M NaOH). The elution was added into a tube containing 200 μL of 0.15 M of acetic acid. Overnight ethanol precipitation was performed at −20° C. and the pellet was collected by centrifugation, washed with 70% ethanol, and dissolved in 15 μL of water; 2 μL of ssDNA was used for PCR amplification, and the rest was stored at −20° C. The selected ssDNA was PCR amplified using PureTaq ready-to-go PCR Beads (GE Healthcare, cat #27-9557-01, Amersham Place, Little Chalfont, Buckinghamshire HP7 9NA, UK) in a volume of 25 μL with 2 μL of 10 uM Forward primer, 2 μL of 10 uM 5′-Biotin Reverse primer, 2 μL ssDNA, and 19 μL H₂O(25 cycles of 30 s at 95° C., 30 s at 50° C., 30 s at 72° C., and finally 5 min at 72° C.). The PCR product was converted to single-stranded DNA by using Dynabeads™ M-280 Streptavidin magnetic beads (ThermoFisher Scientific, cat #11206D, Waltham, MA, USA, standard protocol). The resulting product formed anew enriched library pool that was used for subsequent rounds of SELEX. The process was repeated for nine cycles with increasing selection stringency by increasing the stringency of the washing buffer to 0.5 M NaCl and increasing the wash time. To enhance the specificity of the selected oligonucleotides, counter-selection was performed after the fourth round with thrombin-depleted serum (PrecisionBiologic, cat #FPD02-10, Dartmouth, NS B3B 0A9, Canada) proteins immobilized on magnetic beads. The second counter selection was performed after the sixth round to enrich the library with aptamers that bind to the active site of thrombin. We employed dabigatran, a drug that directly binds to the active site of thrombin, effectively blocking the binding of aptamers to that specific site. Dabigatran (5 nmol) was applied to thrombin beads and blocked thrombin active sites. ssDNA from the sixth round of SELEX was loaded onto the beads and the flow throw was collected. Collected libraries that did not bind to the dabigatran-thrombin complex were subsequently subjected to thrombin bead application, which allowed us to continue the positive selection process, specifically targeting aptamers that exhibited robust binding to thrombin. The SELEX process then proceeded for three more rounds of traditional selection.

### (3) Next-Generation Sequencing of SELEX Samples

134. The single-stranded DNA (ssDNA) obtained after each round of SELEX underwent library preparation for Illumina sequencing using the TruSeq ChIP sample preparation protocol. A total of nine DNA libraries comprised of paired-end indexed sequences were created by pooling the samples. The Illumina TruSeq ChIP sample preparation kit reagents were utilized for this process to facilitate cluster generation and subsequent DNA sequencing. The initial DNA input (50 μL of 200 pg/uL) underwent processes to blunt-end and phosphorylate the molecules. Additionally, a single “A” nucleotide was attached to the 3′ ends of the fragments, preparing them for ligation to adapters featuring a single-base “T” overhang. These adapter sequences were introduced to the DNA ends to generate either indexed single-read or paired-end sequencing libraries. Following ligation, the resulting products were purified and accurately size-selected using agarose gel electrophoresis. The DNA fragments of the desired size were isolated and purified once more. Subsequently, a PCR amplification step was performed to enrich for fragments possessing adapters on both ends. The final product underwent quantification prior to the initiation of cluster generation.

### (4) ELISA-Based Binding Assay of Biotinylated Thrombin Aptamers to Thrombin Protein

135. Native human alpha-thrombin protein (ThermoFisher Scientific, cat #RP-43100, Waltham, MA, USA) was added to each well of a 96-well plate (Nunc MaxiSorp flat-bottom 96-well plates, ThermoFisher Scientific, cat #44-2404-21, Waltham, MA, USA) at a concentration of 250 nM in Tris-buffered saline with a volume of 100 μL per well. The plate was then incubated at 4° C. for 16 h to allow the wells to become coated with thrombin. After removing the thrombin solution, the wells were washed and blocked using blocking buffer (20 mM Tris, 150 mM NaCl pH 7.5, 2% BSA, 0.1% Tween 20 and 100 μg/mL of sheared salmon sperm DNA) for a 1 h duration. Next, biotinylated aptamers at the indicated concentrations in a volume of 100 μL were added to the wells in an incubation buffer (20 mM Tris, 150 mM NaCl pH 7.5, 0.1% BSA, 0.1% Tween 20 and 100 μg/mL of sheared salmon sperm DNA) and incubated for 1 h at room temperature. In the competition binding assay, unlabeled aptamers were added along with the biotinylated aptamers, with the unlabeled aptamers being present in a 100-fold excess compared to the biotinylated aptamers. Following three washing steps with wash buffer (20 mM Tris, 150 mM NaCl pH 7.5, 0.1% BSA and 0.1% Tween 20), streptavidin-HRP (ThermoFisher Scientific, cat #21130, Waltham, MA, USA) was added at a dilution of 1:5000 in the incubation buffer. The plate was washed again three times to remove any unbound reagents. The bound biotinylated aptamers were then detected using 3,3′,5,5′-Tetramethylbenzidine (TMB) substrate (ThermoFisher Scientific, cat #34021, Waltham, MA, USA) following the manufacturer's instructions.

### (5) Secondary Structure Prediction

136. The RNAfold webserver was used to predict the secondary structure of the aptamers. This server is available online and can be used for RNA and ssDNA prediction. Minimum Free Energy (MFE) secondary structure prediction aims to find the structure with the lowest free energy. It predicts the structure by minimizing the thermodynamic free energy of the nucleic acid molecule.

### (6) In Vitro Activity Assay

137. Sterile human blood in sodium citrate was purchased from the Rockland Immuno- chemicals, Inc (cat #R214-0050, Pottstown, PA 19464, USA). Plasma was prepared by centrifugation for 10 min at 2000×g. In vitro activity assays were performed on ACLTOP analyzer manufactured by Instrumentation Laboratory, which is a type of coagulation analyzer commonly used in clinical laboratories to measure clotting times and assess coagulation parameters. The ACLTOP analyzer is designed to perform a wide range of coagulation tests, including Factor II activity, prothrombin time (PT), activated partial thromboplastin time (APTT), fibrinogen, and other related assays. ACLTOP analyzer uses HemosIL reagents developed by Instrumentation Laboratory company (Bedford, MA, USA) to perform coagulation tests.

### (7) Factor II Activity Assay

138. For screening in vitro activity of the aptamers, human citrated plasma (0.5 mL) was incubated in the absence or presence of 2 uM thrombin aptamers or dabigatran for 2 h. Factor II activity was measured using human plasma immunodepleted of Factor II for the quantitative determination of Factor II activity in citrated plasma based on the prothrombin time (PT) assay, for which an ACLTOP coagulation analyzer manufactured by Instrumentation Laboratory (Bedford, MA, USA) was used. To evaluate the dose-dependent activity of the aptamers in whole blood, human blood samples were collected in citrate-treated tubes. Subsequently, the blood samples were incubated at room temperature for 2 h in both the absence and presence of various concentrations of thrombin aptamers. To measure the Factor II activity using the ACLTOP coagulation analyzer, plasma was separated from the cells by centrifugation at 1500×g for 10 min at 4° C. For the dose-dependent study, concentrations of 1 uM, 2 uM, 3 uM, and 4 uM of AYA1809002 and AYA1809004 were utilized.

### (8) Prothrombin Time Assay

139. Human citrated plasma (0.5 mL) was incubated in the absence or presence of 2 uM thrombin aptamers or dabigatran for 2 h. PT time was measured using RecombiPlasTin 2G for quantitative determination in human citrated plasma of PT and Fibrinogen on an ACLTOP coagulation analyzer manufactured by Instrumentation Laboratory (Bedford, MA, USA). To assess the activity of the aptamers in a dose-dependent manner in whole blood, human blood samples were collected in citrate-treated tubes. The blood samples were then incubated at room temperature for 2 h in the absence or presence of varying concentrations of the thrombin aptamers. To measure the Prothrombin Time (PT) on the ACLTOP coagulation analyzer, plasma was obtained by centrifuging the samples at 1500×g for 10 min at 4° C. For the dose dependence study, concentrations of 1 uM, 2 uM, 3 uM, and 4 uM of AYA1809002 and AYA1809004 were utilized.

### (9) Activated Partial Thromboplastin Time (APTT) Assay

140. Human citrated plasma (0.5 mL) was incubated in the absence or presence of 2 uM thrombin aptamers or dabigatran for 2 h. APTT time was measured using the synthetic phospholipid reagent SynthASIil for quantitative determination in human citrated plasma on an ACLTOP coagulation analyzer manufactured by Instrumentation Laboratory (Bed- ford, MA, USA).

### (10) Direct Thrombin Activity Inhibition

141. A Thrombin Inhibitor Screening Assay Kit (fluorometric) (Abcam, cat #ab197007, Waltham, Boston, MA, USA) was employed to test the direct binding ability and inhibitory potential of the AYA1809002 and AYA1809004 aptamers against thrombin. The procedure was carried out following the standard protocol provided with the kit. In brief, aptamers at different concentrations, dabigatran at 2 uM, and controls were added to their respective wells on the plate (ThermoFisher Scientific, cat #3915, Assay Plate, Black, Flat Bottom, Waltham, MA, USA). Subsequently, a thrombin enzyme mixture was added to each sample and control well. Following an incubation period of 20 min, a substrate mixture was introduced into each sample and control well. Fluorescence measurements were taken using a SYNERGY/HTX multi-mode reader (BioTek, Agilent Technologies, Santa Clara, CA 95051, USA) with the excitation/emission wavelengths set at 360/460 nm. The measurements were recorded for 45 min at a temperature of 37° C. while ensuring protection from light.

### (11) Stability Study of Selected Aptamers in Whole Blood at Room Temperature and 37° C.

142. To evaluate the stability of the selected aptamers (AYA1809002, AYA1809004) and compare them to dabigatran, citrated blood collected from a donor was incubated at room temperature in the absence or presence of 1 uM of AYA1809002, AYA1809004, or dabigatran for 0.5, 2, 4, 6, and 24 h. At each time point, blood samples from both the treated and untreated groups were collected and plasma was obtained by centrifugation. The collected plasma samples were then frozen for further analysis. Factor II activity was measured using human plasma immunodepleted of Factor II for quantitative determination of Factor II activity in citrated plasma based on the prothrombin time (PT) assay, for which an ACLTOP coagulation analyzer manufactured by Instrumentation Laboratory was used. PT time was measured using RecombiPlasTin 2G for quantitative determination in human citrated plasma of PT and Fibrinogen on an ACLTOP coagulation analyzer manufactured by Instrumentation Laboratory. The stability experiments at 37° C. were conducted as follows: citrated blood was collected from a donor and incubated with or without 1 uM AYA1809002 or AYA1809004 at 37° C. At each time point, blood samples from both the treated and untreated groups were collected and plasma was obtained by centrifugation. Factor II activity, PT time, and APTT time were measured for each sample. To assess the relative activity, the measured activity of the treated whole blood sample was divided by the activity of the corresponding untreated sample collected at the same time point.

143. To provide additional evidence of the aptamers' stability in an in vitro setting, we conducted the same stability assay using whole blood at 37° C. At various time intervals, we utilized biotinylated reverse complement sequences along with streptavidin-coated magnetic beads to selectively isolate the aptamers from the blood samples. Following elution from the magnetic beads, the aptamers underwent analysis using a Fragment Analyzer system (Agilent Technologies, Santa Clara, CA 95051, USA) to assess their integrity.

(12) Restoration of Factor II Activity and PT Time with a Reverse Complement to AYA1809002 in Whole Blood

144. Citrated blood collected from a donor was incubated in the absence or presence of 1 uM AYA1809002 at room temperature. After 2 h incubation, the indicated concentration of the reverse complement strand was added to the citrated blood sample. After an additional incubation time of 2 h, plasma was obtained through centrifugation. Factor II activity was measured using human plasma immunodepleted of Factor II for the quantitative determination of Factor II activity in citrated plasma based on the prothrombin time (PT) assay, for which an ACLTOP coagulation analyzer manufactured by Instrumentation Laboratory (Bedford, MA, USA) was used. The PT time was measured using RecombiPlasTin 2G for quantitative determination in human citrated plasma of PT and Fibrinogen on an ACLTOP coagulation analyzer manufactured by Instrumentation Laboratory (Bedford, MA, USA). As a control, 1 uM of dabigatran was incubated with the citrated blood.

### (13) Clot-Bound Thrombin

145. For the inhibition study of clot-bound thrombin, blood samples were collected from healthy donors using citrate-treated tubes and centrifuged at 200×g for 15 min at room temperature. The resulting supernatant consisting of platelet-rich plasma (PRP) was collected and aliquots of 500 μL were transferred to microfuge tubes containing 50 μL of 300 mM CaCl₂). The mixture was then incubated for 2 h at 37° C. in an incubator without agitation. The formed clots were washed ten times over the course of 16 h at room temperature; each time, 1 mL aliquots of TBS buffer (50 mM Tris-HCl, 100 mM NaCl, pH 7.4) were added to the clots with mixing. Subsequently, the washed clots were transferred to the assay plate (ThermoFisher Scientific, cat #3915, Waltham, MA, USA). Different concentrations of selected aptamers and 2 μM of dabigatran in 200 μL of TBS buffer were added to their respective wells containing the washed clots. After a 20 min incubation period with agitation, a thrombin substrate (, Thrombin substrate III, Fluorogenic, Sigma-Aldrich, cat #605211, St. Louis, MO, USA) was added to each sample at a final concentration of 200 μM. Fluorescence measurements were performed using a SYNERGY/HTX multi-mode reader (BioTek, Agilent Technologies, Santa Clara, CA 95051, USA) with the excitation/emission wavelengths set at 360/460 nm. The measurements were recorded for a total duration of 90 min at a temperature of 37° C.

### (14) Effect of Modified AYA1809002 and AYA1809004 Aptamers on FACTOR II Activity, PT, and APTT in Human Citrated Plasma and Reversal Potential Using Respective Reverse Complement for Each Aptamer

146. Modified aptamers chemically crosslinked with TAG, PEG, and cholesterol entities at the 3′ end, were purchased from IDT (Integrated DNA Technologies, Coralville, IA, USA). The impact of modified AYA1809002 and AYA1809004 aptamers on Factor II activity, PT, and APTT was evaluated using the same protocol as was used for the unmodified aptamers. Additionally, the potential for reversal was investigated using the respective reverse complement for each aptamer.

(15) Exploring the Immunogenic Reaction of AYA1809002 and AYA1809004 on Human Peripheral Blood Mononuclear Cells (hPBMCs)

147. Human PBMCs were isolated from buffy coats provided by Carter BloodCare(Bedford, TX, USA). Isolation was carried out through density-gradient centrifugation utilizing FicollPaque Plus from GE Healthcare. The isolated human PBMCs suspended in cRPMI media were subjected to stimulation with or without the AYA1809002 and AYA1809004 aptamers and a control aptamer. Various concentrations (1, 5, and 10 uM) of these aptamers were used, along with positive control stimuli such as LPS (200 ng/mL), ODN 1826 (20 uM, Invitrogen), or a combination of LPS (100 ng/mL) and ODN 1826 (10 uM). Following incubation periods of 24 and 72 h, the cell supernatant was collected via centrifugation at 200 g for 3 min. Cytokine levels were evaluated using the LEGENDplex Human Inflammation Panel 1 from BioLegend (cat #740808, San Diego, CA, USA) following the manufacturer's protocol, soluble analytes were acquired using a Navios EX flow cytometer (Beckman Coulter Inc, Pasadena, CA USA), and subsequent analysis was performed using BioLegend's LEGENDPLEX™ system(San Diego, CA, USA).

### (16) Ames Test

148. The mutagenic potential of AYA1809002 and AYA1809004 was evaluated using the Ames test with the Xenometrix Ames MPF PENTA I kit, including S9 and Positive Controls (Aniara Diagnostica, code: ACO1-512-S2-P; manufacturer's part number: C01-512-S2-P, West Chester Township, OH).

### (17) Statistical Analysis

149. Statistical significance was assessed using GraphPad Prism version 10.0.0 (131), 2023 (GraphPad Software, Boston, MA, USA) employing nonparametric methods such as the Mann-Whitney test or unpaired Student's t-test for comparisons between two groups. Data are presented as mean±standard deviation (SD). A p-value of <0.05 was deemed statistically significant, and is indicated by an asterisk (*), while a p-value of <0.01 is denoted by a double asterisk (**).

### b) Results

(1) Developing High-Affinity Neutralizing DNA Aptamers against Human Thrombin Protein

150. To identify single-stranded DNA aptamers capable of binding to human thrombin protein, we employed the SELEX procedure with certain adaptations. This process involves immobilizing human alpha-thrombin protein onto Pierce™ NHS- Activated Magnetic beads following established protocols. It is worth noting that the immobilization process of thrombin on NHS beads involves a random attachment in which lysine (Lys) residues can potentially bind to NHS groups on the beads. In light of the steric challenges posed by the structure of activated thrombin, specifically the deep groove of its active site, utilizing lysine residues located within or near the active site for binding to the beads might be intricate. However, even if such binding occurs, it is important to note that other molecules of thrombin with accessible binding sites remain available for subsequent binding of aptamers.

151. The selection of specific aptamers was carried out using a library of single-stranded DNA oligonucleotides encompassing 10¹⁵ distinct random sequences. These sequences were flanked by two 23-base primer sequences to facilitate selection. In order to enable the unique conformational folding of ssDNA, for the 10 nmol ssDNA library a heating step at 95° C. was followed by cooling on ice. The introduction of bare magnetic beads to the activated ssDNA library was employed to prevent the enrichment of aptamers that solely recognize the beads (negative selection). The SELEX procedure utilized thrombin-coated magnetic beads as the target (FIG. 1). A gradual increase in selection stringency was achieved by raising the wash buffer's NaCl concentration to 0.5 M and increasing the number of wash cycles. After four conventional selection rounds, counter-selection was carried out using magnetic beads immobilized with thrombin-depleted serum proteins. Following the fourth selection round, the eluted ssDNA was subjected to an incubation with thrombin-depleted serum proteins immobilized on magnetic beads. Flow-through was collected for the subsequent selection round after a one-hour incubation. After six rounds of conventional selection, dabigatran was introduced to the thrombin protein immobilized on the beads to block the active site. This complex was employed in order to select those aptamers specifically binding to the active site of thrombin protein (FIG. 1). The eluted ssDNA from the sixth selection round was then incubated with the thrombin- dabigatran complex immobilized on the beads. The flow-through representing aptamers that exclusively bind to the active site of thrombin concealed by dabigatran was collected, then selection proceeded for an additional three rounds. The single-stranded DNA pools eluted from each selection round were chosen for high-throughput sequencing using the Illumina MiSeq platform. Sequence analysis was conducted based on the FASTAptamer method to identify sequences exhibiting enrichment across the various selection pools. The top nine most enriched sequences after nine rounds of selection are listed in Table 1.

TABLE 1
The top nine most enriched sequences after nine rounds of selection.

| Name | Sequence |
|---|---|
| AYA1809001 | GGTAGCGTAAGGATGCGCAAGTTTAATTGCCATATGCCAT (SEQ ID NO: 1) |
| AYA1809002 | GCGGTGTTGCTGGGGTTGGGAGGTTGGAGGAAGCAATCGC (SEQ ID NO: 2) |
| AYA1809003 | GTGTAGGATGGGTGGGTGGGTCACATTTAAGATATCCTGG (SEQ ID NO: 3) |
| AYA1809004 | TCGTCAAGGTACGGTTGGGGGGGTGGGTGTTGACGTTGAA (SEQ ID NO: 4) |
| AYA1809005 | GCGGATGGATGGTGAGGTTGGGAGCTTTCATTGGAACTAA (SEQ ID NO: 5) |
| AYA1809006 | GGGTAGGGTGGTGAGATGAAATCTCTAGGGTGATCGTTCT (SEQ ID NO: 6) |
| AYA1809007 | AGGGGCTCTAGGGTGGGTAGGATGGTGAGACCGCAGCGT (SEQ ID NO: 7) |
| AYA1809008 | CGTGGGGATGGGTGGGTGGAGGAGCGATGATGGCCTACGA (SEQ ID NO: 8) |
| AYA1809009 | GGTAGCGTAAGGATGCGCAAGTTTAATTGCCATATGCCAT (SEQ ID NO: 9) |

### (2) Aptamers Binding to Thrombin

152. The aptamer sequences that displayed the highest enrichment levels after nine rounds of selection were subjected to an ELISA-based binding assay to assess their binding capa- bility to human thrombin. To achieve this, human thrombin protein was immobilized on a MaxiSorp plate, then biotinylated aptamers were introduced and incubated with the immo- bilized protein. Detection of the bound aptamers was accomplished using Streptavidin- HRP and the TMB substrate, with the absorbance measured at 450 nm. Notably, aptamers AYA1809002, AYA1809004, and AYA1809007 exhibited strong binding capabilities, as de- picted in FIG. 2A. To determine the specificity of the aptamers with robust binding affinities, a competition ELISA-based binding assay was employed (FIG. 2B). In this approach, non-biotinylated aptamers in a one-hundred-fold excess relative to their biotinylated counterparts were introduced to the wells containing the respective biotinylated aptamers. If binding is specific, the non-biotinylated aptamers outcompete the biotinylated ones, leading to a significant reduction in absorbance at 450 nm. As demonstrated in FIG. 2B, all three tested aptamers were effectively outcompeted by the corresponding non-biotinylated aptamers in a one-hundred-fold excess, confirming their specific binding to the thrombin protein. The predicted secondary structures of the selected aptamers are displayed in FIG. 2C. For secondary structure prediction, the RNAfold Webserver, available online, was utilized by adapting it for single-stranded DNA prediction. This prediction technique employs the concept of the Minimum Free Energy (MFE) secondary structure to identify the structure with the lowest free energy. The prediction minimizes the thermodynamic free energy of the nucleic acid molecule to forecast its structure. All the chosen aptamers revealed a hairpin structure characterized by unpaired loop sizes ranging from six to eleven nucleotides and MFE values varying from −7.3 kcal/mol to −9.41 kcal/mol (see FIG. 2C). The presence of such a hairpin structure along with unpaired loops in aptamers' secondary structures is important for their stability, binding affinity, specificity, and adaptability to various targets. The loops, which are unpaired regions, can participate in interactions with the target molecule, thereby contributing to the binding affinity and specificity of the aptamer. The loops can fold into structures that mimic the shape and properties of the binding pocket of the target molecule, which can enhance the aptamer's ability to bind tightly to its target by fitting into the binding site with complementary interactions. Importantly, it is worth noting that the selected aptamers are prominently rich in G nucleotides, which implies the potential formation of G quadruplex structures. This structural motif has been identified as being common in many DNA aptamers, and can contribute to the enhanced stability of these aptamers.

(3) In Vitro Assessment of Aptamers to Evaluate their Effectiveness in Modulating the Coagulation Cascade

153. The nine enriched aptamers underwent testing to evaluate their ability to modulate the coagulation cascade and act as direct thrombin inhibitors, similar to dabigatran. They were tested for their ability to inhibit Factor II activity and extend both prothrombin time (PT) and activated partial thromboplastin time (APTT) (FIG. 3). Aptamers AYA1809002, AYA1809004, and AYA1809007 demonstrated superior inhibitory effects on Factor II (FIG. 3A) and the most pronounced efficacy in extending both PT (FIG. 3B) and APTT (FIG. 3C) times. The change of control (untreated condition) served as the baseline, with a normalization of 100%, with the efficacy of the aptamers in altering factor II activity, PT, and APTT presented as a percentage change normalized against the control. At a concentration of 2 uM, AYA1809002 and AYA1809004 exhibit a remarkable reduction in Factor II activity (more than 85%), while AYA1809007 exhibits a decrease in Factor II activity (more than 75%) when compared to Pradaxa (80%). Regarding PT time, AYA1809002 and AYA1809004 demonstrate three-fold increases, while AYA1809007 and dabigatran result in two-fold increases. For APTT, AYA1809002 and AYA1809004 demonstrate an approximate four-fold increase in APTT time, whereas AYA1809007 and dabigatran lead to a three-fold increase. The effects of the aptamers, including the inhibitory effects on Factor II and the extension of PT and APTT time, are comparable to those of dabigatran utilized as a control.

### (4) Binding Affinity of Aptamers AYA1809002 and AYA1809004

154. The combination of binding affinity and in vitro activity data helped to identify the two aptamers AYA1809002 and AYA1809004 as promising candidates for therapeutic applications. The binding affinities of the two most potent aptamers, AYA1809002 and AYA1809004, were assessed using an ELISA-based competition assay. The dissociation constant (Kd) was subsequently determined as well (FIG. 4). The assay involved incubating the indicated concentrations of either 5′ Bioitn-AYA1809002 (FIG. 4A) or 5′ Biotin-AYA1809004 (FIG. 4B) with thrombin protein immobilized on a 96-well ELISA plate. This was done in the absence or presence of a 100-fold excess of non-biotinylated AYA1809002 or AYA1809004, respectively. Absorbance was measured after incubation with streptavidin-horseradish peroxidase bound to the biotinylated aptamer in the presence of TMB substrate. The estimated binding affinity of AYA1809002 to thrombin was 10 nM, while that of AYA1809004 was 13 nM.

### (5) Direct Thrombin Activity Inhibition by AYA1809002 and AYA1809004 in a Dose-Dependent Manner

155. The direct binding and inhibitory capabilities of AYA1809002 and AYA1809004 ap- tamers on thrombin were evaluated using a Thrombin Inhibitor Screening Assay Kit (fluorometric). The procedure was carried out following the standard protocol provided with the kit. The protocol includes incubating the samples with thrombin followed by the addition of a substrate. The fluorescence intensity is then measured over time to assess the thrombin inhibitory potential of the sample. The kit utilizes the enzymatic activity of thrombin to hydrolyze a synthetic substrate based on AMC (7-amino-4-methylcoumarin), resulting in the liberation of AMC. The released AMC is detectable through fluorescence measurement at an excitation/emission wavelength of 360/460 nm. When thrombin-specific inhibitors are present, the degree of cleavage reaction is diminished or entirely suppressed. As depicted in FIG. 5, aptamer AYA1809002 (FIG. 5A) and aptamer AYA1809004 (FIG. 5B) demonstrated dose-dependent inhibition of thrombin activity. This provides confirmation that the aptamers both bind directly to thrombin and effectively inhibit its enzymatic activity.

### (6) Dose-Dependent Effect of AYA1809002 and AYA1809004 on Factor II Activity and PT (Prothrombin Time) in Whole Blood

156. To facilitate future applications, gaining a comprehensive understanding of the activity of aptamers in whole blood is of paramount importance. To assess their effectiveness, we tested the selected aptamers at various concentrations in whole blood to evaluate their impact on Factor II activity and PT time. Human blood samples were collected in citrate- treated tubes. The blood samples were then incubated at room temperature for 2 h in the absence or presence of varying concentrations of thrombin aptamers. To measure Factor II activity and PT time on the ACLTOP coagulation analyzer, plasma was obtained by centrifuging the samples at 1500× g for 10 min at 4° C. For the dose dependence study, concentrations of 1 uM, 2 uM, 3 uM, and 4 uM of AYA1809002 and AYA1809004 were utilized. FIG. 6 illustrates a clear dose-dependent decrease in Factor II activity for both aptamers (FIG. 6A,C). Additionally, as the concentration of the aptamers increases there is a corresponding increase in prothrombin time (FIG. 6B,D). Dabigatran at a concentration of 1 uM was used as a control. These results demonstrate a dose-dependent effect of AYA1809002 and AYA1809004 on both Factor II activity and PT time, indicating that the selected aptamers impact the overall coagulation process.

### (7) Stability of Selected Aptamers in Whole Blood

157. The stability of aptamers in whole blood holds significant importance for their future applications, as it provides insights into their longevity and potential as anticoagulants or thrombin inhibitors compared to the reference drug dabigatran. To evaluate the stability of the selected aptamers AYA1809002 and AYA1809004 and compare them to dabigatran, citrated blood collected from a donor was incubated at room temperature in the absence or presence of 1 uM of AYA1809002, AYA1809004, or dabigatran for 0.5, 2, 4, 6, and 24 h. At each time point, blood samples from both the treated and untreated groups were collected and plasma was obtained through centrifugation. As depicted in FIG. 7, AYA1809002 and AYA1809004 demonstrated stability and effectiveness for up to 24 h. This finding indicates that the aptamers retained their inhibitory properties against thrombin during the entire 24 h period of incubation. By performing the same experiment using whole blood at 37° C. for 24 h, we aimed to evaluate the stability and effectiveness of the aptamers in vitro under physiological conditions, specifically at body temperature (FIG. 13). The fact that the effectiveness of the aptamers remained intact for the entire 24 h duration at 37 C indicates that the aptamers are stable in vitro and capable of exerting their anticoagulant effects even under conditions that mimic the human body's physiological temperature.

158. To further substantiate the stability of the aptamers in an in vitro environment, we replicated the stability assay using whole blood at 37° C. Across different time points, we employed biotinylated reverse complement sequences in conjunction with streptavidin- coated magnetic beads to carefully extract the aptamers from the blood samples. Upon elution from the magnetic beads, the aptamers were subjected to analysis using a fragment analyzer. The outcomes of this investigation are depicted in FIG. 16. The traces of the aptamers at various time intervals confirm that the aptamers remain structurally intact even after 24 h of incubation in citrated blood at 37° C. Recognizing that this study represents a preliminary phase in assessing the stability of these aptamers in an in vivo context, in order to enhance the durability of the aptamers within the blood- stream we integrated the well-established stabilizing agents PEG, TEG, and cholesterol into our aptamer designs. In summary, our methodology encompassed a comprehensive assessment of aptamer stability in vitro, combining coagulation cascade modulation with analysis of their structural integrity in whole citrated blood at physiologically relevant temperatures.

(8) Restoration of Factor II Activity and PT Time with Reverse Complement to AYA1809002 in Whole Blood

159. The ability to reverse or counteract the anticoagulant effects of thrombin inhibitors is essential in situations where immediate hemostasis is required, such as during emergency surgeries, major bleeding events, or in patients who experience complications associated with anticoagulant therapy. One approach to reverse the anticoagulant effect of aptamers is to use their reverse complement sequences. The reverse complement sequence is designed to bind to and neutralize the aptamer's activity. To investigate the ability of the reverse complement strand to reverse the anticoagulant effect of AYA1809002, citrated blood collected from a donor was incubated in the absence or presence of 1 uM AYA1809002 at room temperature. After 2 h, the indicated concentration of the reverse complement strand was added to the citrated blood sample with the aptamer. After an additional incubation period, plasma was collected per the existing protocol and Factor II activity (FIG. 8A) and PT time (FIG. 8B) were measured. Based on FIG. 8, it is evident that the reverse complements of the aptamers completely inhibit their activity and effectively restore both Factor II activity and PT time even at a 1:1 ratio with the aptamers. The reverse complements act as antagonists to the aptamers, effectively reversing their impact on the coagulation cascade.

### (9) Clot-Bound Thrombin

160. Clot-bound thrombin refers to thrombin that becomes localized and trapped within a blood clot. In this state, it continues to promote fibrin formation, resulting in the further growth and stabilization of the clot. To ensure comprehensive evaluation of the selected aptamers, it is essential to test their inhibitory potential against thrombin when it is in the clot-bound stage. For the inhibition study of clot-bound thrombin, we utilized the method previously described in with a few modifications. Clots were generated in human platelet-rich plasma by adding CaCl₂) and incubating for 2 h at 37° C. After washing the clots, they were transferred to TBS buffer containing either AYA1809002, AYA1809004, or dabigatran. Following a brief incubation, thrombin fluorogenic substrate was added to each sample and fluorescence measurements were conducted. Using this approach, we employed the ability of clot-bound thrombin to cleave a synthetic AMC-based substrate to release AMC. Based on the data presented in FIG. 9, both aptamers demonstrated the ability to inhibit clot-bound thrombin in a concentration-dependent manner, with aptamer AYA1809002 exhibiting a higher level of inhibition compared to AYA1809004. Notably, the inhibition of clot-bound thrombin with the two aptamers is comparable to that of dabigatran.

### (10) Modified AYA1809002 and AYA1809004 Aptamers

162. To enhance their stability in the bloodstream, the selected aptamers were chemically modified through crosslinking with TEG, PEG, and cholesterol moieties (Table 2).

TABLE 2
Modified and Control Aptamer Sequences Used in the Study.

| Modified Aptamers | Sequences |
|---|---|
| 3′PEG AYA1809002 | GCGGTGTTGCTGGGGTTGGGAGGTTGGAGGAAGCAATCGC/3Sp18/ |
| | (SEQ ID NO: 16) |
| 3′TEG AYA1809002 | GCGGTGTTGCTGGGGTTGGGAGGTTGGAGGAAGCAATCGC/3Sp9/ |
| | (SEQ ID NO: 17) |
| 3′Chol AYA1809002 | GCGGTGTTGCTGGGGTTGGGAGGTTGGAGGAAGCAATCGC/3CholTEG/ |
| | (SEQ ID NO: 18) |
| 3′PEG AYA1809004 | TCGTCAAGGTACGGTTGGGGGGGTGGGTGTTGACGTTGAA/3Sp18/ |
| | (SEQ ID NO: 19) |
| 3′TEG AYA1809004 | TCGTCAAGGTACGGTTGGGGGGGTGGGTGTTGACGTTGAA/3Sp9/ |
| | (SEQ ID NO: 20) |
| 3′Chol AYA1809002 | TCGTCAAGGTACGGTTGGGGGGGTGGGTGTTGACGTTGAA/3CholTEG/ |
| | (SEQ ID NO: 21) |
| Control Aptamer | GCGCGGTCCCGATTTGGTGTAAAATTCCCTCAGCCCTACA |
| | (SEQ ID NO: 22) |

The modified AYA1809002 and AYA1809004 aptamers were then evaluated to determine their impact on Factor II activity, PT, and APTT in plasma. Additionally, the potential reversal of their effects was investigated using reverse complement sequences that correspond to each aptamer (FIGS. 14 and 15). FIG. 14 clearly illustrates that the modified AYA1809002 exhibits a similar impact on Factor II, PT, and APTT compared to the unmodified aptamers. Notably, when the reverse complement is applied it effectively reverses the observed effects. The modified AYA1809004 aptamer is less effective compared to the non-modified aptamer (FIG. 15). The cholesterol-modified AYA1809004 showed no significant impact on Factor II activity, PT, or APTT. However, the PEG- and TEG-modified variants demonstrated noticeable inhibition of Factor II activity and a slight increase in PT and APTT times. The reverse complement of AYA1809004 effectively restored the impact on Factor II activity, PT, and APTT for all modified aptamers, indicating that the reverse complement sequence can counteract the inhibitory effects observed with the modified aptamer.

### (11) Safety of the AYA1809002 and AYA1809004 Aptamers

163. Aptamers are generally considered to have a good safety profile thanks to their bio- compatibility and similarity to naturally occurring nucleic acids. The evaluation of aptamer safety involves assessing their stability, specificity, immunogenicity, and mutagenicity.

### (12) Immunogenicity Assessment of AYA1809002 and AYA1809004 Aptamers.

164. To investigate the immunogenicity of AYA1809002 and AYA1809004 using an in vitro human model, we measured the levels of human inflammatory cytokines and chemokines induced by varying doses of AYA1809002 and AYA1809004 in cultured human peripheral blood mononuclear cells (PBMCs). The assessed cytokines included IL-1β, IFN-α2, IFN-γ, TNF-α, MCP-1 (CCL2), IL-6, IL-8 (CXCL8), IL-10, IL-12p 70, IL-17A, IL-18, IL-23, and IL-33. The PBMCs were subjected to the indicated conditions, and the culture media were collected at 24 and 72 h post-treatment, as illustrated in FIG. 10. For human PBMCs treated with 1, 5, or 10 uM concentrations of AYA1809002 and AYA1809004, the secretion of IL-1β, IFN-α2, IFN-γ, TNF-α, MCP-1 (CCL2), IL-6, IL-8 (CXCL8), IL-10, IL-12p70, IL-17A, IL-18, IL-23, and IL-3 was comparable to that of cells treated with mock or control aptamers at both 24 and 72 h. The positive control groups, especially a combination of LPS (100 ng/mL) and ODN 1826 (10 uM), exhibited elevated production of IL-10, IFN-u2, IFN-γ, TNF-α, IL-6, IL-8 (CXCL8), IL-10, IL-12p70, IL-17A, IL-18, IL-23, and IL-3 as compared to mock-treated cells at both 24 and 72 h following stimulation at the specified doses. Collectively, these findings indicate that AYA1809002 and AYA1809004 do not exhibit immune-stimulating properties. These observations demonstrated that a smooth muscle cell-targeted RNA aptamer did not induce increased release of the inflammatory cytokines IL-6, IFN-j3, or IFN-γ from human PBMCs. Based on our investigations, AYA1809002 and AYA1809004 seem to possess a favorable safety profile.

(13) Mutagenicity Assessment of AYA1809002 and AYA1809004 Aptamers using the Ames Test

165. To assess the mutagenicity of the aptamers, we performed Ames test, a widely used assay for evaluating the mutagenicity of chemical compounds. The Ames test is a bac-terial reverse mutation assay that detects the ability of a substance to induce mutations in the DNA of certain strains of bacteria. It utilizes various bacterial strains, including Salmonella typhimurium strains TA98, TA100, TA1535, and TA1537, as well as the Escherichia coli strains wp2 [pKM101]and wp2 uvrA. To evaluate the mutagenic potential of an aptamer, amino acid-requiring bacterial organisms are exposed to different concentrations of the aptamer and mutagenic events are assessed by selecting for reversion events. In this context, the aptamers AYA1809002 and AYA1809004 were supplemented at increasing concentrations ranging from 0.5 uM to 10 uM. Our observations revealed that all tested strains treated with various concentrations of the aptamers exhibited fewer revertants compared to the established baseline represented by the positive control cut-off (see FIGS. 11 and 12). Taken together, these findings collectively indicate that the aptamers AYA1809002 and AYA1809004 do not exhibit mutagenic properties.

### c) Discussion

166. Thrombin plays a crucial role as a central orchestrator in the blood coagulation cascade, encompassing various essential functions such as pro-coagulation, anticoagulation, platelet aggregation, and inflammatory activities. From hirudin, to parenteral direct thrombin inhibitors (lepirudin, desirudin, argatroban, and bivalirudin), to oral direct thrombin inhibitors (ximelagatran and dabigatran), scientists have been working hard to investigate and develop a range of thrombin inhibitors in recent decades. However, the use of thrombin inhibitors is limited or contradicted in clinics due to drug interactions, side effects, risk of bleeding, and cost. The biggest challenge in developing thrombin inhibitors is the need to reduce bleeding-related risks while maintaining high anti-thrombin efficacy. Therefore, developing a thrombin inhibitor that is efficient, safe, affordable, and has minimal side effects remains highly necessary. In this study, we employed the Systematic Evolution of Ligands by Exponential Enrichment (SELEX) method to generate a diverse range of aptamers targeting the active site of thrombin. Notably, during the SELEX procedure we introduced dabigatran as a competitor to enhance the selection specificity.

167. Employing this approach, we successfully obtained a pool of aptamers that dis-played high affinity and selectivity toward thrombin. Within this enriched pool, aptamers AYA1809002 and AYA1809004 demonstrated the highest binding affinity for thrombin among all of the identified candidates. The effectiveness of these aptamers was evaluated in vitro using an FDA-approved ACLTOP instrument in both plasma and whole blood samples. The assessment focused on measuring Factor II activity, PT time (Prothrombin Time), and APTT time (Activated Partial Thromboplastin Time). The results of the study demonstrated a clear dose-dependent effect of both aptamers on Factor II activity. As the concentration of the aptamers increased, there was a corresponding reduction in Factor II activity. This indicates that the aptamers effectively inhibited the functionality of Factor II, a key component of the coagulation cascade. Furthermore, the study revealed an increase in both PT time and APTT time with increasing concentrations of the aptamers. PT time is a measure of the extrinsic pathway of the coagulation cascade, while APTT time assesses the intrinsic pathway. These prolonged PT and APTT times indicate that the aptamers impacted the coagulation process and led to a delay in clot formation. These results support the utility of the selected aptamers as anticoagulant agents or tools for studying the coagulation process.

168. Extensive research has been conducted over the years to find reversal agents for anticoagulation therapy. Numerous studies have focused on identifying and evaluating effective strategies to reverse the anticoagulant effects of various anticoagulants, including thrombin inhibitors. The unique properties of aptamers allow for the rational design of antidotes, providing a means to extend the heparin-protamine paradigm to a new class of direct-acting specific anticoagulants. This drug-antidote design technology offers the possibility of novel antidote-based control of antithrombotic activity. As a result, several studies have focused on the development of anticoagulation therapies by selecting DNA aptamers that specifically bind to thrombin, as mentioned earlier. The ability of the reverse complements of the AYA1809002 and AYA1809004 aptamer sequences to restore Factor II activity and PT time provides valuable insights into the regulatory mechanisms involved in the coagulation process. In addition, it highlights the ability to develop therapeutic strategies utilizing reverse complements to counteract the effects of aptamers when necessary. The findings presented in FIG. 8 indicate the ability of reverse complements to modulate and fine-tune the coagulation processes by counteracting the effects of aptamers.

169. Chemical modifications such as crosslinking with TEG, PEG, or cholesterol entities were employed to enhance the stability of the aptamers in the bloodstream. Remarkably, these modifications did not compromise the effectiveness of the aptamers in terms of inhibiting Factor II activity and prolonging PT and APTT times. The modified aptamers retained their inhibitory effects on the measured coagulation parameters, demonstrating their potential as stable and functional candidates for anticoagulation therapy. Ap- tamers AYA1809002 and AYA1809004 were shown to be safe, as they demonstrated a non-immunogenic nature. This indicates a favorable safety profile for these molecules, making them well-tolerated in therapeutic applications. Additionally, a mutagenicity assessment, specifically the Ames test, confirmed that these aptamers do not possess mutagenic properties. These findings provide strong evidence supporting the safety and biocompatibility of AYA1809002 and AYA1809004, reinforcing their use therapeutic interventions. It is worth noting that the selected aptamers are rich in G. This observation indicates the potential formation of G quadruplex structures, a prevalent motif found in numerous DNA aptamers. This structural characteristic can enhance the stability of these aptamers. When Bock and Toole discovered the first single-strand DNA aptamer against human protease thrombin, they discovered that the sequence of GGTTGG (SEQ ID NO: 13) was the most conserved sequenced in almost all of their selected clones. Greatly delayed thrombin-catalyzed conversion of fibrinogen to fibrin has been observed at 15 mer with sequences 5′-GGTTGGTGTGGTTGG-3′ (SEQ ID NO: 10). Using this aptamer, Li et al. demonstrated its efficiency to inhibit clot-bound thrombin activity and reduce arterial platelet thrombus formation. Among the enriched aptamers, with the exception of AYA1809001, there were similarities in the sequence motif GGTTGG (SEQ ID NO: 13). AYA1809002 possessed the extended sequence GGTTGGGAGGTTGG (SEQ ID NO: 14), while AYA1809004 had the longer sequence GGTTGGGGGGGTTGG (SEQ ID NO: 15). These sequences are G-rich and have the potential to form G-quadruplex structures, similar to the aptamers studied by Bock and Toole. The selected aptamers exhibit a predicted hairpin structure with unpaired loop sizes ranging from six to eleven nucleotides. The presence of such a hairpin structure, along with unpaired loops in the aptamers' secondary structures, is important for their stability, binding affinity, specificity, and adaptability to various targets. Hairpin structures can provide conformational flexibility, allowing the aptamer to adapt its structure in order to more optimally interact with the target. This adaptability is crucial for effective binding, as the target molecule can have different conformations or binding requirements. The fact that multiple aptamers share a similar structural feature (a hairpin with unpaired loops) indicates that there can be a common binding mechanism or principle that underlies their interactions with their respective targets.

170. The selected aptamers were subjected to testing to evaluate their ability to inhibit both fluid-phase thrombin and clot-bound thrombin. This comprehensive assessment allowed us to determine their efficacy in targeting thrombin in different contexts. By inhibiting fluid- phase thrombin, the selected aptamers can directly prevent excessive thrombin activity in the blood, while their ability to inhibit clot-bound thrombin offers the ability to prevent further thrombin-mediated clotting events and inhibit the propagation of the clot. This dual inhibitory action further highlights their versatility and therapeutic value in managing thrombotic conditions.

171. In this study, we have demonstrated the properties and anticoagulant activity of two anti-thrombin aptamers. The findings indicate that AYA1809002 and AYA1809004 can be effective, safe, and affordable therapies for thrombosis.

### d) Conclusions

172. The primary goal in developing these new anti-thrombin DNA aptamers is to broaden the selection of aptamer candidates that can be used for clinical applications. Our comprehensive investigation has revealed compelling evidence supporting the efficacy and safety of AYA1809002 and AYA1809004 as potent anti-thrombin candidates. These aptamers demonstrate remarkable performance in vitro in inhibiting thrombosis, effectively countering excessive clot formation, and their activities are easily reversible by administration of the antidote. Furthermore, a comprehensive assessment of their safety profile through in vitro studies demonstrated minimal negative impacts, reinforcing their use in clinical applications.

### 2. Example 2: Treatment of thrombosis

173. Thrombosis is a critical medical condition characterized by the formation of blood clot(s) within a blood vessel- artery or a vein. If left untreated, the clot has the potential to impede, obstruct, or migrate within the bloodstream, leading to severe life-threatening conditions such as thromboembolic stroke, heart attack, disseminated intravascular coagulation (DIC), venous thromboembolism (VTE), or pulmonary embolism (PE). Statistics indicate that thrombosis is the leading cause of death worldwide, being responsible for one in four deaths. In the United States, approximately 900,000 people are affected by VTE or PE annually, with an estimated 25% (approx. 225,000) facing sudden death. Over the past few years, there has been a gradual shift in the approach to treating thrombosis, transitioning from traditional anticoagulants (heparin and vitamin K antagonists) to the use of direct oral anticoagulants (DOACs). While use of traditional coagulants is limited due to its parenteral route of administration, the most striking concern with the current DOACs is the potential risk of causing life-threatening internal bleeding conditions. In addition, they also have severe side effects, drug interactions, shorter half-life, high costs of production and the lack of an antidote to reverse the anticoagulant effects. Moreover, the fixed dosing regimen of DOACs can disadvantageous in patients with renal impairment, elderly patients, or those with extreme body weights. Thus, there exists a pressing need for novel anticoagulant agents with high affinity and specificity, stability, favourable safety profile, ability to reverse the anticoagulant effect, low costs, and improved patient outcomes, that can surmount the limitations of existing options. The aptamers disclosed bind and inhibits thrombin, and offers a promising treatment strategy for thrombosis. With experimentally demonstrated improved anticoagulant efficacy, favorable safety profile, high affinity and reversibility through the administration of an antidote ex vivo, AYA1809002 aptamer promises a precise, effective, safe, stable, controllable and reversible therapy with low costs of production, signifying a new generation of antithrombotic agents that can address the unmet needs of current DOACs.

174. The current medications of thrombosis pose several disadvantages including the potential risk of causing life-threatening internal bleeding, severe side effects, high costs of production and lack of antidote to reverse the effects as needed clinically during bleeding emergencies or before urgent procedures. The aptamers disclosed herein offer a precise, effective, safe, stable, controllable, and cost-effective thrombosis therapy reversible with a reverse aptamer. This novel treatment would lead to better management of thrombosis and hence prevent severe life-threatening conditions such as thromboembolic stroke, heart attack, venous thromboembolism (VTE), disseminated intravascular coagulation (DIC), or pulmonary embolism (PE).

175. What is the rationale behind the dosing range (in mg kg) ofAYA1809002?The proposed dosing range is extrapolated from the demonstrated efficacious ex vivo concentration (2 μM) to account for bioavailability, distribution, metabolism, and excretion in vivo. The lower dose (0.8 mg/kg) represents the minimum effective dose, the mid-range (1.6 mg/kg) can help assess dose-dependent responses, and the higher dose (4 mg/kg) can explore the upper safety limit and efficacy plateau. This broad range ensures comprehensive evaluation of AYA1809002's efficacy and safety. The volume of 100 μL has been standardized to be appropriate for safe administration in small rodents.

176. The DNA aptamer, AYA1809002, inhibits thrombin with high anticoagulant activity, reversibility, and safety, offering a promising thrombosis treatment. Details are in the Innovation section. Based on the known properties of aptamers, the therapy is expected to benefit: i) high bleeding risk individuals with a history of gastrointestinal bleeding or at risk for intracranial hemorrhage, ii) patients requiring frequent anticoagulation adjustments for perioperative management or invasive procedures, iii) patients undergoing intermittent hemodialysis or other procedures requiring cyclical anticoagulation, and iv) Heparin-Induced Thrombocytopenia (HIT) patients. The precise subset of users can be more clearly identified after evaluating efficacy and safety.

177. The focus of the present disclosure isn't just on competing in niche markets like other aptamers that focus on HIT patients. Instead, the focus is to provide a next-generation broad spectrum anticoagulant that can revolutionize thrombosis treatment (The anticoagulant and antidote can be distributed separately. Similar to currently used antidotes like Pradaxa (idarucizumab), our antidote aptamer can likely need to be administered in medical facilities. This ensures proper dosing, monitoring, and management of potential complications in emergency situations, such as severe bleeding or urgent surgery needs.

178. Thrombosis is a critical medical condition characterized by the formation of blood clot(s) within a blood vessel, be it an artery or a vein. If left untreated, the clot has the potential to impede, obstruct, or migrate within the bloodstream, leading to severe life-threatening conditions such as thromboembolic stroke, heart attack, venous thromboembolism (VTE), disseminated intravascular coagulation (DIC), or pulmonary embolism (PE). Statistics indicate that thrombosis is the leading cause of death worldwide, being responsible for one in four deaths^{2,3}. In the United States, ˜900,000 people are affected by VTE or PE annually, with an estimated 25% (˜225,000) facing sudden death⁴. Furthermore, among individuals who have experienced a deep vein thrombosis (DVT), one third to one half have lasting complications known as post-thrombotic syndrome, including symptoms such as swelling, pain, discoloration, and scaling in the affected limb. Moreover, within a span of 10 years, around one-third (˜33%) of individuals with DVT or PE are likely to encounter a recurrence⁴. Over the past few years, there has been a gradual shift in the approach to treating thrombosis, transitioning from traditional anticoagulants with the limitation of parenteral route of administration to the use of direct oral anticoagulants (DOACs). However, the most striking concern with the current DOACs is the potential risk of causing life-threatening internal bleeding conditions⁵. In addition, they also pose several disadvantages including severe side effects, drug interactions, shorter half-life and high costs of production⁵. Moreover, the fixed dosing regimen of DOACs can be a disadvantage in patients with renal impairment, elderly patients, or those with extreme body weights. Thus, there exists a pressing need for novel anticoagulant agents with increased affinity and activity, stability, favorable safety profile, ability to reverse the anticoagulant effect, low costs, and improved patient outcomes, that can surmount the limitations of existing options.

179. The present disclosure provides groundbreaking advancement in thrombosis therapy by developing a novel aptamer, AYA1809002, that binds and inhibits thrombin, a key enzyme that plays a central orchestrating role in procoagulation, anticoagulation, and platelet activation. AYA1809002 demonstrates heightened anticoagulant efficacy and safety with high affinity, specificity and reversibility through the administration of an antidote. Therefore, AYA1809002 aptamer promises precise, effective, safe, stable, controllable and reversible therapy with low costs of production, signifying a new generation of antithrombotic agents that can address the unmet needs of current DOACs.

a) In vivo evaluation of optimal dose, efficacy and immunotoxicity of aptamer.

180. Non-modified and PEG-modified AYA1809002 can be evaluated for their anticoagulant effects by measuring coagulation markers (thrombin time (TT), Factor II activity, prothrombin time (PT) and activated partial thromboplastin time (APTT) (1.1), plasma pharmacokinetic profile (1.2), and immunotoxicity (1.3) in collected blood, urine and organs samples..

### B) In Vivo Efficacy and Immunotoxicity of Aptamer and Antidote in Clot Induction Assay.

181. Clot formation can be induced in rats by injection of collagen and epinephrine intravenously. The top aptamer with optimum concentration selected from Aim 1 and its antidote can be tested for its anticoagulant effects (2.1). The efficacy (2.1) and immunotoxicity (2.2) of the antidote can also be tested. Additionally, assessments on downstream embolic diseases can be carried out.

182. Our novel AYA1809002 aptamer can revolutionize thrombosis therapy by offering a highly specific, effective, safe, stable, controllable, reversible and cost-effective antithrombotic agent. This is bound to have a positive impact on management of thrombosis and is well aligned with the NHLBI's objectives of developing and optimizing novel diagnostic and therapeutic strategies to prevent, treat, and cure HLBS diseases, to improve people's health and QOL.

### c) SIGNIFICANCE

183. Thrombosis is a critical medical condition characterized by the formation of blood clot(s) within a blood vessel- artery or a vein. If left untreated, the clot has the potential to impede, obstruct, or migrate within the bloodstream, leading to severe life-threatening conditions such as thromboembolic stroke, heart attack, disseminated intravascular coagulation (DIC), venous thromboembolism (VTE), or pulmonary embolism (PE). Statistics indicate that thrombosis is the leading cause of death worldwide, being responsible for one in four deaths. In the United States, approximately 900,000 people are affected by VTE or PE annually, with an estimated 25% (approximately 225,000) facing sudden death⁴. Furthermore, among individuals who have experienced a deep vein thrombosis (DVT), one third to one half have lasting complications known as post-thrombotic syndrome, including symptoms such as swelling, pain, discoloration, and scaling in the affected limb. Moreover, within a span of 10 years, around one-third (approximately 33%) of individuals with DVT or PE are likely to encounter a recurrence. The current therapeutic options for thrombosis are inadequate. Over the past few years, there has been a gradual shift in the approach to treating thrombosis, transitioning from traditional anticoagulants (heparin and vitamin K antagonists) to the use of direct oral anticoagulants (DOACs) (oral direct thrombin inhibitors (DTIs), oral factor Xa inhibitors). While use of traditional coagulants is limited due to its parenteral route of administration, the most striking concern with the current DOACs is the risk of causing life-threatening bleeding conditions such as intracranial hemorrhage (ICH) and gastrointestinal bleeding (GIB). In addition, they also have severe side effects, drug interactions (cytochrome P450 system (3A4) and Pglycoprotein (P-gp), shorter half-life, high costs of production and lack of specific agent to reverse anticoagulant effects. Moreover, the fixed dosing regimen of DOACs can be a disadvantage in patients with renal impairment, elderly patients, or those with extreme body weights as the one-size-fits-all approach to dosing can lead to either underdosing or overdosing, which may result in decreased efficacy or increased risk of bleeding, respectively. Thus, there exists a pressing need for novel anticoagulant agents with high affinity and specificity, stability, favorable safety profile, ability to reverse the anticoagulant effect, low costs, and improved patient outcomes, that can surmount the limitations of existing options.

184. The coagulation cascade involves the activation of a series of clotting factors, which are proteins that are involved in blood clotting. Each clotting factor is a serine protease, an enzyme that speeds up the breakdown of another protein. Thrombin is a key enzyme that plays a central orchestrating role in procoagulation, anticoagulation, and platelet activation. In procoagulation, thrombin converts fibrinogen into insoluble fibrin clots while activating platelets and factors XIII, V, VIII, and XI. In anticoagulation, thrombin plays a regulatory and control role in the coagulation cascade by activating protein C, which together with its cofactor protein S degrades FVIIIa and FVa. The diverse functions of thrombin are largely attributed to its intricately dynamic three-dimensional molecular structure. Thrombin comprises a catalytic site along with two anion-binding active sites known as exosite I and exosite II. Exosite I serves as thrombin's primary active site, facilitating interactions with a range of molecules such as fibrinogen, fibrin, heparin cofactor II, and protease-activated receptor. Exosite II, on the other hand, serves as the specific site responsible for activating factors V and VIII, as well as for binding with heparin

185. Recently, aptamers have emerged as promising tools for targeted treatment of various diseases. Aptamers are short single-stranded (ss) oligonucleotides that consist of either RNA or DNA that have high specificity and affinity to their target molecules. Generated through the Systematic Evolution of Ligands by Exponential Enrichment (SELEX) method, these nucleic acid-based modalities are known for their small size, stability, low immunogenicity, programmability, versatility, low costs of production, and holding potential for diagnosis, prophylaxis, and treatment. Moreover, aptamers' flexible three-dimensional structures allow them to fold around the complex surfaces of their target molecules, such as peptides, proteins, small organic compounds, toxins, cells, viruses, bacteria, etc. Hence, aptamers can be used as therapeutic agents to improve the therapeutic index and overall health impact. There are few types of therapeutic aptamers that have already been approved by the US FDA for disease treatment. Pegaptanib is the first aptamer that is approved by the FDA for the treatment of age-related macular degeneration. Thus, aptamer-based drugs offer promising therapeutic treatments for thrombosis. In one aspect, disclosed herein is the DNA aptamer, AYA1809002, that binds and inhibits thrombin, and offers a promising treatment strategy for thrombosis.

### (1) Targeting of Thrombin Protein

186. AYA1809002 specifically binds to the active site of the thrombin protein with high binding affinity. The active site of thrombin is positioned between exosite 1 and exosite 2, forming a functional triad. The key molecular mechanism of action for AYA1809002 is its specific and reversible binding to the active site cleft within the protease domain of the thrombin protein, selectively inhibiting access to the catalytic triad and blocking thrombin's enzymatic activity in the coagulation cascade, while minimizing off-target effects and adverse reactions. By selectively binding and inhibiting thrombin, AYA1809002 is highly effective at reducing thromboembolic events. This specificity also minimizes off-target effects, reducing the risk of adverse reactions commonly associated with broader anticoagulant therapies.

### (2) High Anticoagulant Activity and Reversibility

187. AYA1809002 demonstrates potent anticoagulant activity in vitro coupled with a unique built-in antidote system (reverse aptamer) to rapidly reverse effects as needed clinically during bleeding emergencies or before urgent procedures (Table 3). This is the first demonstration of thrombin aptamer with reversible function, overcoming a major limitation of prior candidates. This drug-antidote design technology offers the possibility of novel antidote-based control of antithrombotic activity.

TABLE 3
Competitive advantages of Ayass Bioscience's aptamer
over existing solutions

| | Direct oral | Ayass Bioscience's |
|---|---|---|
| | anticoagulant (DOACs, | AYA1809002 DNA |
| Criteria | et. Dabigatran) | aptamer |
| Increased Affinity | IC50 for thrombin | IC50 for thrombin |
| and Activity | inhibition ~4.5 nM | inhibition ≤0.5 nM |
| Specificity | ~300-fold selectivity | >100-fold selectivity |
| | | for thrombin over |
| | | other serine proteases |
| Safety | Life-threatening | Low immunogenicity |
| | bleeding risks (′ 3.3% | and side effects |
| | in clinical trials) | (≤2% major bleeding |
| | | rate in animal |
| | | studies) |
| Pharmacokinetics | Half-life of 12-17 | Half-life of 12-15 |
| | hours but with high | hours with low |
| | variability [Coefficient | variability [CV <20%] |
| | of Variation | |
| | (CV) ~40%] | |
| Onset of Action | Peak effect 2-3 hours | Peak effect withing 1 hour |
| Renal Clearance | ~80% | <50% |
| Anticoagulant | ~30% variability | <15% inter-individual |
| Effect Consistency | | variability in |
| | | aPTT prolongation |
| Reversal of | Idarucizumab achieves | Complete reversal |
| Anticoagulant | this in ~2-4 | within 10 minutes of |
| Effect | hours | antidote administration |
| Manufacturing | Higher Production costs | Projected 30% lower |
| Costs | Less stable, | per dose and it is |
| (Anticoagulant | requires cold chain | chemically synthesized |
| and | storage and | and scalable |
| antidote) | transportation | Developing a |
| | | synthesis process |
| | | with ≥90% yield to |
| | | reduce waste and |
| | | cost |
| | | Targeting a shelf |
| | | life of ≥2 years |
| | | to minimize supply |
| | | chain costs |
| | | Demonstrating |
| | | feasibility of |
| | | scaling production |
| | | to >1 kg per batch |
| Stability | Requires refrigeration, | Maintain >95% |
| | shelf life 36 | activity after 24 months |
| | months | at room temperature |

### (3) Modification

188. AYA1809002 aptamer has been advanced in optimization through chemical modification by crosslinking with polyethylene glycol (PEG) moiety to prolong plasma half-life while retaining function.

### (4) Advantages over competing solutions

189. The disclosure provided herein represents a highly innovative approach in the development of controllable and reversible anticoagulant to improve thrombosis treatment (Table 3). This aptamer-based drug-antidote can provide more treatment flexibility compared to current options, adapting to various clinical scenarios and offering versatile approaches. The significantly smaller size of antidote aptamer offers a faster onset of reversal action, which is critical in emergency situations and high-risk procedures and a more complete reversal due to better tissue penetration. In comparison to conventional anticoagulants and DOACs, AYA1809002 being short and single-stranded nucleic acid molecule, offers a safer alternative as they demonstrate no observed mutagenic or immunogenic effects. This enhanced safety provides a more tolerable treatment option for patients undergoing thrombosis therapy, making it suitable for broader patient populations and long-term use. Due to the small size of AYA1809002 aptamer, it can be reproducibly manufactured at low costs, offering high scalability. Therefore, with demonstrated heightened anticoagulant efficacy, favorable safety profile, high affinity and reversibility through the administration of an antidote, AYA1809002 aptamer promises precise, effective, safe, stable, controllable and reversible therapy with low costs of production, signifying a new generation of antithrombotic agents that can address the unmet needs of current DOACs.

190. AYA1809002 can revolutionize thrombosis therapy. This highly specific, effective, safe, stable, controllable, reversible and cost-effective antithrombotic agent can significantly have a positive impact on patients' health and quality of life. The development of AYA1809002 aptamer is well aligned with the National Heart, Lung, and Blood Institute's (NHLBI) objectives of developing and optimizing novel diagnostic and therapeutic strategies to prevent, treat, and cure HLBS diseases.

### d) RESULTS

191. Ayass Bioscience has attained data of its technology through extensive evaluation of its DNA aptamers. We have conducted in vitro and ex vivo studies to scrutinize the binding affinity and specificity to thrombin, anticoagulant effects and safety to show AYA1809002's ability as therapeutic agent against thrombosis¹.

192. Primarily, single-stranded DNA aptamers capable of binding to human thrombin protein were identified by employing the SELEX procedure. Notably, during the SELEX process, Dabigatran Etexilate (Pradaxa; an FDA-approved direct and reversible thrombin inhibitor) was introduced as a competitor to enhance the selection specificity. After nine rounds of selection, the top nine aptamer sequences that displayed the highest enrichment levels were selected and subjected to an ELISA-based binding assay to assess their binding capability to human thrombin (FIG. 2A). They were evaluated for their ability to modulate the coagulation cascade and act as direct thrombin inhibitors, similar to dabigatran. Based on the data of the initial screening experiments of binding affinity and in vitro anticoagulation activity, the aptamer AYA1809002 was selected as the most promising candidate for therapeutic applications.

### (1) AYA1809002 has Optimal Structure and Binding Affinity.

193. AYA1809002 revealed a hairpin structure characterized by unpaired loop sizes ranging from six to eleven nucleotides and Minimum Free Energy (MFE) was valued at -8.72 kcal/mol (FIG. 2C). The presence of such a hairpin structure along with unpaired loops in aptamers' secondary structures is important for their stability, binding affinity, specificity, and adaptability to various targets. The binding affinity of AYA1809002 was assessed using an ELISA-based competition assay. The dissociation constant (Kd) was subsequently determined as well (FIG. 4A). The assay involved incubating the indicated concentrations of 5′ Bioitn-AYA1809002 with thrombin protein immobilized on a 96-well ELISA plate in the absence or presence of a 100-fold excess of non-biotinylated AYA1809002. Absorbance was measured after incubation with streptavidin horse radish peroxidase bound to the biotinylated aptamer in the presence of the TMB substrate. Each bar is an average of duplicate measurements. The binding affinity of AYA1809002 to thrombin was estimated to be 10 nM (FIG. 3B).

### (2) AYA1809002 Directly Affects Thrombin Activity, Factor II Activity and PT (Prothrombin Time) in Whole Blood.

194. The direct binding and inhibitory capabilities of AYA1809002 on thrombin were evaluated using a Thrombin Inhibitor Screening Assay Kit (fluorometric). The kit utilizes the enzymatic activity of thrombin to hydrolyze a synthetic substrate resulting in the liberation of AMC (7-amino-4-methylcoumarin). The released AMC is detectable through fluorescence measurement at an excitation/emission wavelength of 360/460 nm. When thrombin-specific inhibitors are present, the degree of cleavage reaction is diminished or entirely suppressed. AYA1809002 demonstrated dose-dependent inhibition of thrombin activity, confirming that it bound directly to thrombin and effectively inhibited its enzymatic activity (FIG. 5A). To further assess the effectiveness in anticoagulatory activity, AYA1809002 was tested for its ability to inhibit Factor II activity and extend prothrombin time (PT) in whole blood in a dose-dependent manner. Human blood samples were collected in citrate-treated tubes. The blood samples were then incubated at RT for 2 h in the absence or presence of varying concentrations (1 μM, 2 μM, 3 μM, and 4 μM) of thrombin aptamer. To measure Factor II activity and PT time on the ACLTOP coagulation analyzer, plasma was obtained by centrifuging the samples at 1500×g for 10 min at 4° C. A clear dose-dependent decrease in Factor II activity was illustrated (FIG. 6A). Additionally, as the concentration of AYA1809002 increased, there was a corresponding increase in PT (FIG. 6B). Dabigatran (1 μM) was used as a control. These results demonstrated a dose-dependent effect on both Factor II activity and PT time, indicating that AYA1809002 impacts the overall coagulation process and performs better than Dabigatran.

### (3) AYA1809002 Inhibits Clot-Bound Thrombin.

195. Clot-bound thrombin refers to thrombin that becomes localized and trapped within a blood clot. In this state, it continues to promote fibrin formation, resulting in the further growth and stabilization of the clot. AYA1809002 demonstrated the ability to inhibit clot-bound thrombin in a concentration-dependent manner.

### (4) AYA1809002 do not Exhibit Immunogenic and Mutagenic Properties.

196. To investigate the immunogenicity of AYA1809002 using an in vitro human model, the levels of human inflammatory cytokines and chemokines induced by varying doses of AYA1809002 were measured in cultured human peripheral blood mononuclear cells (PBMCs). AYA1809002 did not exhibit immune-stimulating properties. Further, to assess the mutagenicity of the aptamer, Ames test was performed which revealed that AYA1809002 did not exhibit mutagenic properties.

### (5) Reverse Complement of AYA1809002 Restores Factor II Activity and PT Time in Whole Blood.

197. The ability to reverse or counteract the anticoagulant effects of thrombin inhibitors is essential in situations where immediate hemostasis is required, such as during emergency surgeries, major bleeding events, or in patients who experience complications associated with anticoagulant therapy. To reverse the anticoagulant effect of the aptamer, the reverse complement sequence of AYA1809002 was designed to bind to and neutralize the aptamer's activity. To validate this, citrated blood collected from a donor was incubated in the absence or presence of 1 μM AYA1809002 at RT. After 2 h, the indicated concentrations (1:1, 1:3 and 1:5) of the reverse complement strand was added to the citrated blood sample with the aptamer. After an additional incubation period, plasma was collected as per the existing protocol and Factor II activity and PT time were measured. The reverse complements of AYA1809002 completely inhibited the activity and effectively restored both Factor II activity and PT time even at a 1:1 ratio with the aptamers (FIG. 8). Therefore, the reverse complements act as antagonists to the aptamers, effectively reversing their impact on the coagulation cascade.

### (6) Modified AYA1809002 Aptamer and Reverse Aptamer.

198. To enhance the stability in the bloodstream, AYA1809002 was chemically modified through crosslinking with TEG, PEG, and cholesterol moieties. The modified AYA1809002 effectively reversed the observed effects on Factor II, PT, and activated partial thromboplastin time (APTT) and were compared to the unmodified aptamers.

### (7) AYA1809002 and PEG-Modified AYA1809002 Inhibit Factor II Activity and Extend PT, APPT and TT in Rat Whole Blood.

199. For further preclinical validations of safety and efficacy of AYA1809002 in an animal model, it's binding affinity to rat thrombin was measured using a Biolayer Interferometry (BLI) assay on the GatorPrime system. AYA1809002 was biotinylated and immobilized on streptavidin-coated biosensor tips. The biosensors were then exposed to varying concentrations of rat thrombin protein, allowing real-time monitoring of thrombin binding to the aptamer. Kinetic parameters were derived by fitting the BLI binding curves to a 1:1 binding model. The dissociation constant (KD) that quantifies the binding affinity was estimated to be -0.6 nM to rat thrombin (FIG. 17). Moreover, AYA1809002 and PEG-modified AYA1809002 were able to inhibit Factor II activity and extend PT, APPT and TT in rat whole blood in a dose-dependent manner. The reverse complement strand of AYA1809002 neutralized the observed effects. AYA1809002 and PEG-modified AYA1809002 were capable of specifically binding to thrombin protein and effectively impacting the coagulation cascade in human as well as rat plasma. Their reverse aptamers were also able to neutralize the coagulation effects. Therefore, AYA1809002 and PEG-modified AYA1809002 are promising candidates for the development of novel anticoagulants.

### e) METHODS

(1) Aptamer administration.

200. Rats can be administered with a single intravenous (IV) bolus of AYA1809002 (0.8 mg/kg, 1.6 mg/kg, 4 mg/kg; 100 ptL) via lateral tail vein. The dosing is selected based on ex vivo data on human and rat plasma. Control group can be administered with a PBS solution. Sample collection. Blood samples (0.3 mL) can be collected from tail snip before and at predefined time points post-administration of aptamer (0h, 1h, 4h, 8h, and 24h) and plasma from blood samples can be isolated as described before¹. Urine samples can also be collected at 0h, 1h, 4 h, 8h, and 24h. Two animals (one male and one female) can be euthanized at the 4h time point and tissues including lung, brain, kidney, liver, spleen, bladder, bone marrow, and urinary tract can also be collected. At the end of the study (24h timepoint), all animals can be euthanized, and tissues mentioned above can be collected.

### (2) Determination of Plasma Pharmacokinetic Profile.

201. To analyze AYA1809002's absorption, distribution and excretion, quantitative real-time RT-PCR (RT-qPCR) assays can be performed on the collected blood, tissues and urine samples respectively. We can isolate AYA1809002 from the samples by designing a biotin labelled reverse complement sequence. This probe can then be introduced to the sample under conditions that enable hybridization between the probe and its complementary aptamer target. The biotinylated probe-aptamer hybrid construct can then be pulled down using streptavidin-coated magnetic beads. The eluted aptamer can be subjected to qPCR analysis. Data can be analyzed to determine key pharmacokinetic parameters, such as clearance, volume of distribution, half-life, and area under the curve (AUC).

(3) Assessment of immunotoxicity.

202. To demonstrate safety of AYA1809002, immunogenic responses can be studied by investigation of the production of 79 inflammatory cytokines (including IL-1β, IFN-α2, IFN-γ, TNF-α, MCP-1 (CCL2), IL-6, IL-8 (CXCL8), IL-10, IL-12p70, IL-17A, IL-18, IL-23, and IL-33) by animals using the Biotechne Proteome Profiler Rat XL Cytokine Array multiplex assay using the FLEXMAP 3D instrument by Luminex. To evaluate safety, a comprehensive panel assessing kidney function via blood markers and urine protein/glucose, liver function through serum liver enzymes, bilirubin, and liver histopathology; and gastrointestinal effects by monitoring diarrhea, food/water intake changes, electrolyte imbalance, and stool character can be conducted.

### (4) Animal Model.

203. Rats (Sprague Dawley rats, 6-8 weeks old, 200-250g) can be used. A total of 24 animals (1:1, male:female; plus overage of 8 animals) can be used.

(5) Clot induction assay.

204. Clot formation can be induced in rats by IV injection of collagen and epinephrine. While collagen stimulates platelet activation and aggregation, epinephrine accelerates the clotting cascade. Combining the two agents reliably triggers systemic coagulation. Aptamer administration. After 2 h of clot induction (this allows the formed clot to stabilize and reach a more mature state before the therapy is introduced), rats of group #2 and #4 can be administered with a single IV bolus dose of AYA1809002 (top aptamer with best concentration identified herein, 100 ptL) via lateral tail vein. Control group #1 can be administered with a PBS solution.

(a) Sample collection.

205. Blood samples (approximately 0.3 mL each) can be collected from two animals in treatment groups #1 and #2, one male and one female, at 4 h after clot induction. This 4-hour timepoint allows for 2 hours of clot formation and an additional 2 hours post-treatment to assess the efficacy of the aptamer. The collected blood can be evaluated for coagulation markers.

### (6) Antidote Administration.

206 After 2 h, rats of group #3 and #4 can be administered with a single IV bolus dose of AYA1809002 antidote (3-folds) via lateral tail vein. Control group #1 can be administered with a PBS solution.

### (a) Sample Collection.

207. Blood samples (0.3 mL) can be collected before and at predefined time points post-administration of antidote (0h, 1 min, 5 min, 15 min, 30 min and 60 min) and plasma from blood samples can be isolated. At the end of the study (60 min timepoint), animals of group #1, #3 and #4 can be euthanized, and tissues (lung, brain, kidney, liver, spleen, bladder, bone marrow, and urinary tract) can be collected.

### (7) Assessment of Anticoagulant Effects of Aptamer and Antidote.

208. To assess efficacy of the aptamer to prevent further clot formation and the antidote to reverse the observed effect, post clot induction, anticoagulant effects including aPTT, PT, TT and Factor II activity can be measured ex vivo in collected blood samples, using the ACLTOP coagulation analyzer.

### (8) Assessment of Immunotoxicity of Antidote.

209. To demonstrate safety of AYA1809002 antidote, immunogenic responses and potential toxicities in key organ systems can be studied.

### (9) Assessments of Downstream Embolic Diseases.

210. Cardiac histopathology, ECG monitoring and evaluation of cardiac biomarkers including cardiac troponins, CKMB, and hs-CRP can be carried out.

### E. Sequences

(SEQ ID NO: 1)
GGTAGCGTAAGGATGCGCAAGTTTAATTGCCATATGCCAT
(SEQ ID NO: 2)
GCGGTGTTGCTGGGGTTGGGAGGTTGGAGGAAGCAATCGC
(SEQ ID NO: 3)
GTGTAGGATGGGTGGGTGGGTCACATTTAAGATATCCTGG
(SEQ ID NO: 4)
TCGTCAAGGTACGGTTGGGGGGGGGGTGTTGACGTTGAA
(SEQ ID NO: 5)
GCGGATGGATGGTGAGGTTGGGAGCTTTCATTGGAACTAA
(SEQ ID NO: 6)
GGGTAGGGTGGTGAGATGAAATCTCTAGGGTGATCGTTCT
(SEQ ID NO: 7)
AGGGGCTCTAGGGTGGGTAGGATGGTGAGACCGCAGCGT
(SEQ ID NO: 8)
CGTGGGGATGGGTGGGTGGAGGAGCGATGATGGCCTACGA
(SEQ ID NO: 9)
GGTAGCGTAAGGATGCGCAAGTTTAATTGCCATATGCCAT
(SEQ ID NO: 10)
GGTTGGTGTGGTTGG
(SEQ ID NO: 11)
AGTCCGTGGTAGGGCAGGTTGGGGTGACT
(SEQ ID NO: 12)
TAGGGAAGAGAAGGACATATGAT(N40)TTGACTAGTACATGACCAC
TTGA
(SEQ ID NO: 13)
GGTTGG
(SEQ ID NO: 14)
GGTTGGGAGGTTGG
(SEQ ID NO: 15)
GGTTGGGGGGGTTGG
(SEQ ID NO: 16)
GCGGTGTTGCTGGGGTTGGGAGGTTGGAGGAAGCAATCGC/3Sp18/
(SEQ ID NO: 17)
GCGGTGTTGCTGGGGTTGGGAGGTTGGAGGAAGCAATCGC/3Sp9/
(SEQ ID NO: 18)
GCGGTGTTGCTGGGGTTGGGAGGTTGGAGGAAGCAATCGC/
3CholTEG/
(SEQ ID NO: 19)
TCGTCAAGGTACGGTTGGGGGGGGGGTGTTGACGTTGAA/3Sp18/
(SEQ ID NO: 20)
TCGTCAAGGTACGGTTGGGGGGGTGGGTGTTGACGTTGAA/3Sp9/
(SEQ ID NO: 21)
TCGTCAAGGTACGGTTGGGGGGGTGGGTGTTGACGTTGAA/
3CholTEG/
(SEQ ID NO: 22)
GCGCGGTCCCGATTTGGTGTAAAATTCCCTCAGCCCTACA
(SEQ ID NO: 23)
GCGATTGCTTCCTCCAACCTCCCAACCCCAGCAACACCGC
(SEQ ID NO: 24)
TTCAACGTCAACACCCACCCCCCCAACCGTACCTTGACGA

### F. References

Alam, K.; Chang, J.; Burke, D. FASTAptamer: A bioinformatic toolkit for high-throughput sequence analysis of combinatorial selections. Mol.-Ther.-Nucleic Acids 2015, 4, e230.
Ansell, J.; Bakhru, S.; Laulicht, B.; Steiner, S.; Grosso, M.; Brown, K.; Dishy, V.; Noveck, R.; Costin, J. Use of PER977 to reverse the anticoagulant effect of edoxaban. N. Engl. J. Med. 2014, 371, 2141-2142.
Ayass, M.; Griko, N.; Pashkov, V.; Tripathi, T.; Cao, W.; Javan, N.; Dai, J.; Zhang, J.; Zhu, K.; Abi-Mosleh, L. High-Affinity Neutralizing DNA Aptamers against SARS-CoV-2 Spike Protein Variants. COVID 2023, 3, 520-542.
Ayass, M.; Tripathi, T.; Griko, N.; Pashkov, V.; Dai, J.; Zhang, J.; Herbert, F.; Ramankutty, Nair, R.; Okyay, T.; Zhu, K.; et al. Highly efficacious and safe neutralizing DNA aptamer of SARS-CoV-2 as an emerging therapy for COVID-19 disease. Virol. J. 2022, 19, 227.
Bhatia, K.; Ladd, L.; Carr, K.; Di Napoli, M.; Saver, J.; McCullough, L.; Hosseini, Farahabadi, M.; Alsbrook, D.; Hinduja, A.; Ortiz, Garcia, J.; et al. Contemporary Antiplatelet and Anticoagulant Therapies for Secondary Stroke Prevention: A Narrative Review of Current Literature and Guidelines. Curr. Neurol. Neurosci. Rep. 2023, 23, 235-262.
Blommel, M.; Blommel, A. Dabigatran etexilate: A novel oral direct thrombin inhibitor. Am. J Health-Syst. Pharm. 2011, 68, 1506-1519.
Bock, L.; Griffin, L.; Latham, J.; Vermaas, E.; Toole, J. Selection of single-stranded DNA molecules that bind and inhibit human thrombin. Nature 1992, 355, 564-566.
Bruno, J. G. Predicting the uncertain future of aptamer-based diagnostics and therapeutics. Molecules 20, 6866-6887 (2015) Burn, J. & Pirmohamed, M. Direct oral anticoagulants versus warfarin: Is new always better than the old?Open Heart 5, (2018).
Cera, E. Thrombin as procoagulant and anticoagulant. J. Thromb. Haemost. 2007, 5, 196-202.
Chadha, A., Guirguis, M. & Bungard, T. J. DOAC drug interactions management resource. Canadian Pharmacists Journal 155, 315-325 (2022).
Chaudhry, R.; Usama, S.; Babiker, H. Physiology, Coagulation Pathways; StatPearls Publishing, Treasure Island, FL USA 2018.
Chen, A.; Stecker, E.; Warden, B.A. Direct oral anticoagulant use: A practical guide to common clinical challenges. J. Am. HeartAssoc. 2020, 9, e017559.
Condrey, J.; Flietstra, T.; Nestor, K.; Schlosser, E.; Coleman-McCray, J.; Genzer, S.; Welch, S.; Spengler, J. Prothrombin time, activated partial thromboplastin time, and fibrinogen reference intervals for inbred strain 13/n guinea pigs (Cavia porcellus) and validation of low volume sample analysis. Microorganisms 2020, 8, 1127.
Derszniak, K.; Przyborowski, K.; Matyjaszczyk, K.; Moorlag, M.; De Laat, B.; Nowakowska, M.; Chlopicki, S. Comparison of effects of anti-thrombin aptamers HD1 and HD22 on aggregation of human platelets, thrombin generation, fibrin formation, and thrombus formation under flow conditions. Front. Pharmacol. 2019, 10, 68.
Di Cera, E. Thrombin as procoagulant and anticoagulant. Journal of Thrombosis and Haemostasis 5, 196-202 (2007) Ferguson, L.; Denny, W. The genetic toxicology of acridines. Mutat. Res. Genet. Toxicol. 1991, 258, 123-160.
Gast, A.; Tschopp, T.; Schmid, G.; Hilpert, K.; Ackermann, J. Inhibition of clot-bound and free (fluid-phase thrombin) by a novel synthetic thrombin inhibitor (Ro 46-6240), recombinant hirudin and heparin in human plasma. Blood Coagul. Fibrinolysis Int. J Haemost. Thromb. 1994, 5, 879-887.
Gopinath, S. Anti-coagulant aptamers. Thromb. Res. 2008, 122, 838-847.
Hakura, A.; Shimada, H.; Nakajima, M.; Sui, H.; Kitamoto, S.; Suzuki, S.; Satoh, T. Salmonella/human S9 mutagenicity test: A collaborative study with 58 compounds. Mutagenesis 2005, 20, 217-228.
Hughes, Q.; Le B.; Gilmore, G.; Baker, R.; Veedu, R. Construction of a bivalent thrombin binding aptamer and its antidote with improved properties. Molecules 2017, 22, 1770.
Kotkowiak, W.; Pasternak, A. Beyond g-quadruplexes—The effect of junction with additional structural motifs on aptamers properties. Int. J. Mol. Sci. 2021, 22, 9948.
Kretz, C.; Stafford, A.; Fredenburgh, J.; Weitz, J. HD1, a thrombin-directed aptamer, binds exosite 1 on prothrombin with high affinity and inhibits its activation by prothrombinase. J. Biol. Chem. 2006, 281, 37477-37485.
Kubo, T.; Urano, K.; Utsumi, H. Mutagenicity characteristics of 255 environmental chemicals. J Health Sci. 2002, 48, 545-554.
Kulabhusan, P. K., Hussain, B. & Yuce, M. Current perspectives on aptamers as diagnostic tools and therapeutic agents. Pharmaceutics 12, 1-23 (2020).
Lane, D.; Philippou, H.; Huntington, J. Directing thrombin. Blood 2005, 106, 2605-2612.
Levy, J.; Ageno, W.; Chan, N.; Crowther, M.; Verhamme, P.; Weitz, J. When and how to use antidotes for the reversal of direct oral anticoagulants: Guidance from the SSC of the ISTH. J Thromb. Haemost. 2016, 14, 623-627.
Li, Z. et al. Advances in Screening and Development of Therapeutic Aptamers Against Cancer Cells. Front Cell Dev Biol 9, (2021).
Li, M.; Li, J.; Wang, X.; Hui, X.; Wang, Q.; Xie, S.; Yan, P.; Tian, J.; Li, J.; Xie, P.; et al. Oral direct thrombin inhibitors or oral factor Xa inhibitors versus conventional anticoagulants for the treatment of pulmonary embolism. Cochrane Database Syst. Rev. 2023.
Liu, M., Zaman, K. & Fortenberry, Y. M. Overview of the therapeutic potential of aptamers targeting coagulation factors. Int J Mol Sci 22, (2021).
Mallett, S.; Cox, D. Thrombelastography. Br. J Anaesth. 1992, 69, 307-313.
Malovichko, M.; Sabo, T.; Maurer, M. Ligand binding to anion-binding exosites regulates conformational properties of thrombin. J Biol. Chem. 2013, 288, 8667-8678.
Mazurov, A.; Titaeva, E.; Khaspekova, S.; Storojilova, A.; Spiridonova, V.; Kopylov, A.; Dobrovolsky, A. Characteristics of anew DNA aptamer, direct inhibitor of thrombin. Bull. Exp. Biol. Med. 2011, 150, 422-425.
Mocini, D. et al. Direct oral anticoagulants in patients with obesity and atrial fibrillation: Position paper of italian national association of hospital cardiologists (ANMCO). J Clin Med 10, (2021).
Moldovan, M.; Filip, L.; Ciurea, M.; Termure, D.; Ostas, D.; Rotar, H.; Faur, C.; Roman, R. Bleeding and thromboembolic risk in patients under anticoagulant therapy receiving oral surgery: A systematic review. Med. Pharm. Rep. 2023.
Müller, J.; Freitag, D.; Mayer, G.; Pötzsch, B. Anticoagulant characteristics of HD1-22, a bivalent aptamer that specifically inhibits thrombin and prothrombinase. J Thromb. Haemost. 2008, 6,2105-2112.
Murphy, M., Fuller, S., Richardson, P. & Doyle, S. An improved method for the in vitro evolution of aptamers and applications in protein detection and purification. Nucleic Acids Research. 31, e110-el10 (2003)
Nagano, M.; Kubota, K.; Sakata, A.; Nakamura, R.; Yoshitomi, T.; Wakui, K.; Yoshimoto, K. A neutralizable dimeric anti-thrombin aptamer with potent anticoagulant activity in mice. Mol.- Ther.-Nucleic Acids 2023, 26, 1-9.
Padmanabhan, K.; Padmanabhan, K.; Ferrara, J.; Sadler, J.; Tulinsky, A. The structure of alpha-thrombin inhibited by a 15-mer single-stranded DNA aptamer. J Biol. Chem. 1993, 268, 17651-17654.
Petrera, N.; Stafford, A.; Leslie, B.; Kretz, C.; Fredenburgh, J.; Weitz, J. Long range communication between exosites 1 and 2 modulates thrombin function. J Biol. Chem. 2009, 284, 25620-25629.
Pollack, C., Jr.; Reilly, P.; Eikelboom, J.; Glund, S.; Verhamme, P.; Bernstein, R.; Dubiel, R.; Huisman, M.; Hylek, E.; Kamphuisen, P.; et al. Idarucizumab for dabigatran reversal. N. Engl. J. Med. 2015, 373, 511-520.
Ponce, A. T. & Hong, K. L. A mini-review: Clinical development and potential of aptamers for thrombotic events treatment and monitoring. Biomedicines 7, (2019).
Prival, M.; Zeiger, E. Chemicals mutagenic in Salmonella typhimurium strain TA1535 but not in TA100. Mutat. Res. Toxicol. Environ. Mutagen. 1998, 412, 251-260.
Rasche, H. Haemostasis and thrombosis: An overview. Eur. Heart J. Suppl. 2001, 3, Q3-Q7.
Rusconi, C.; Scardino, E.; Layzer, J.; Pitoc, G.; Ortel, T.; Monroe, D.; Sullenger, B. RNA aptamers as reversible antagonists of coagulation factor IXa. Nature 2002, 419, 90-94.
Soule, E.; Bompiani, K.; Woodruff, R.; Sullenger, B. Targeting two coagulation cascade proteases with a bivalent aptamer yields a potent and antidote-controllable anticoagulant. Nucleic Acid Ther. 2016, 26, 1-9.
Stoll, H.; Steinle, H.; Wilhelm, N.; Hann, L.; Kunnakattu, S.; Narita, M.; Schlensak, C.; Wendel, H.; Avci-Adali, M. Rapid complexation of aptamers by their specific antidotes. Molecules 2017, 22, 954.
Tasset, D.; Kubik, M.; Steiner, W. Oligonucleotide inhibitors of human thrombin that bind distinct epitopes. J Mol. Biol. 1997, 272, 688-698.
Thiel, W.; Esposito, C.; Dickey, D.; Dassie, J.; Long, M.; Adam, J.; Streeter, J.; Schickling, B.; Takapoo, M.; Flenker, K.; et al. Smooth muscle cell-targeted RNA aptamer inhibits neointimal formation. Mol. Ther. 2016, 24, 779-787.
Troisi, R.; Napolitano, V.; Spiridonova, V.; Russo, Krauss, I.; Sica, F. Several structural motifs cooperate in determining the highly effective anti-thrombin ractivity of NU172 aptamer. Nucleic Acids Res. 2018, 46, 12177-12185.
Tucker, W. O.; Shum, K. T.; Tanner, J. A. G-quadruplex DNA aptamers and their ligands: Structure, function and application. Curr. Pharm. Des. 2012, 18, 2014-2026.
Vahle, J. Safety assessment including current and emerging issues in toxicologic pathology. In Haschek and Rousseaux's Handbook of Toxicologic Pathology, 3rd ed.; Elsevier: Amsterdam, The Netherlands, 2013; pp. 1051-1073.
Van Ryn, J.; Hauel, N.; Waldmann, L.; Wienen, W. Dabigatran Inhibits Both Clot-Bound and Fluid Phase Thrombin In Vitro: Effects Compared to Heparin and Hirudin. Blood 2007, 110, 3998.
Van Ryn, J.; Stangier, J.; Haertter, S.; Liesenfeld, K.; Wienen, W.; Feuring, M.; Clemens, A. Dabigatran etexilate-A novel, reversible, oral direct thrombin inhibitor: Interpretation of coagulation assays and reversal of anticoagulant activity. Thromb. Haemost. 2010, 103, 1116-1127.
Venkatesan, S., Chanda, K. & Balamurali, M. M. Recent Advancements of Aptamers in Cancer Therapy. ACS Omega 8, 32231-32243 (2023).
Wei, S. et al. An update on applications and limitations of direct oral anticoagulants. Egypt J Intern Med 35, (2023)
Wendelboe, A. M. & Raskob, G. E. Global Burden of Thrombosis: Epidemiologic Aspects. Circ Res 118, 1340-1347 (2016)
White, R.; Rusconi, C.; Scardino, E.; Wolberg, A.; Lawson, J.; Hoffman, M.; Sullenger, B. Generation of species cross-reactive aptamers using “toggle” SELEX. Mol. Ther. 2001, 4, 567-573.

Woodruff, R.; Sullenger, B. Modulation of the coagulation cascade using aptamers. Arterioscler. Thromb. Vasc. Biol. 2015, 35, 2083-2091.
Yuminova, A.; Smirnova, I.; Arutyunyan, A.; Kopylov, A.; Golovin, A.; Pavlova, G. The structure of G-quadruplex thrombine- binding DNA aptamer RA36. Mosc. Univ. Chem. Bull. 2015, 70, 43-46.
Li, W.; Kaplan, A.; Grant, G.; Toole, J.; Leung, L. A novel nucleotide-based thrombin inhibitor inhibits clot-bound thrombin and reduces arterial platelet thrombus formation. Blood 1994, 83, 677-682.

What is claimed is:

1. An isolated nucleic acid comprising the sequence as set forth in SEQ ID NO: 1, SEQ ID NO: 7, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21, or any fragment or variant thereof comprising at least 90% sequence identity thereto.

2. The isolated nucleic acid of claim 1, wherein the nucleic acid comprises sequence as set forth in SEQ ID NO: 2 or SEQ ID NO: 4.

3. The RNA equivalent of any of the isolated nuclei acids of claim 1.

4. The isolated nucleic acid of claim 1, further comprising a detectable tag.

5. The isolated nucleic acid 4, wherein the detectable tag comprises a latex bead, magnetic bead, fluorescence label; fluorescent probe, chemiluminescent labels, radiolabels, and/or nanoparticle probe.

6. The isolated nucleic acid of claim 1, further comprising a stabilizing moiety.

7. The isolated nucleic acid of claim 1, wherein the stabilizing moiety comprises polyethylene glycol (PEG), triethylene glycol (TEG), or cholesterol.

8. A pharmaceutical composition comprising one or more of the isolated nucleic acids of claim 1.

9. A kit comprising one or more of the isolated nucleic acids of claim 1.

10. The kit of claim 9, further comprising an aptamer antidote.

11. The kit of claim 10, wherein the aptamer antidote comprises SEQ ID NO: 23 or SEQ ID NO: 24.

12. The kit of claim 10, wherein the isolated nucleic acid comprises SEQ ID NO: 2, SEQ ID NO: 16, SEQ ID NO: 17, and/or SEQ ID NO: 18 and the aptamer antidote comprises SEQ ID NO: 23.

13. The kit of claim 10, wherein the isolated nucleic acid comprises SEQ ID NO: 4, SEQ ID NO: 19, SEQ ID NO: 20, and/or SEQ ID NO: 21 and the aptamer antidote comprises SEQ ID NO: 24.

14. A method of treating thrombosis or a thrombotic event in a subject in need thereof comprising administering to the subject one or more of the isolated nucleic acids of claim 1.

15. The method of claim 14, wherein the thrombosis or thrombotic event comprises stroke, heart attack, deep vein thrombosis (DVT), disseminated intravascular coagulation (DIC), venous thromboembolism (VTE), or pulmonary embolism (PE).

16. An aptamer antidote comprising the sequence as set forth in SEQ ID NO: 23 or SEQ ID NO: 24.

17. A method of treating adverse events from administration of an aptamer therapy comprising administering to a subject having previously received the aptamer therapy, the aptamer antidote of claim 16.

18. The method of claim 17, wherein the aptamer comprises SEQ ID NO: 2, SEQ ID NO: 16, SEQ ID NO: 17, and/or SEQ ID NO: 18 and the aptamer antidote comprises SEQ ID NO: 23.

19. The method of claim 17, wherein the aptamer comprises SEQ ID NO: 4, SEQ ID NO: 19, SEQ ID NO: 20, and/or SEQ ID NO: 21 and the aptamer antidote comprises SEQ ID NO: 24.


SEQUENCE LISTING (24 sequences)

SEQ ID NO: 1 - DNA, 40 bp, synthetic construct
  misc_feature 1..40: note = Description of sequence: aptamer
  source 1..40: mol_type = other DNA
ggtagcgtaaggatgcgcaagtttaattgccatatgccat

SEQ ID NO: 2 - DNA, 40 bp, synthetic construct
  misc_feature 1..40: note = Description of sequence: aptamer
  source 1..40: mol_type = other DNA
gcggtgttgctggggttgggaggttggaggaagcaatcgc

SEQ ID NO: 3 - DNA, 40 bp, synthetic construct
  misc_feature 1..40: note = Description of sequence: aptamer
  source 1..40: mol_type = other DNA
gtgtaggatgggtgggtgggtcacatttaagatatcctgg

SEQ ID NO: 4 - DNA, 40 bp, synthetic construct
  misc_feature 1..40: note = Description of sequence: aptamer
  source 1..40: mol_type = other DNA
tcgtcaaggtacggttgggggggtgggtgttgacgttgaa

SEQ ID NO: 5 - DNA, 40 bp, synthetic construct
  misc_feature 1..40: note = Description of sequence: aptamer
  source 1..40: mol_type = other DNA
gcggatggatggtgaggttgggagctttcattggaactaa

SEQ ID NO: 6 - DNA, 40 bp, synthetic construct
  misc_feature 1..40: note = Description of sequence: aptamer
  source 1..40: mol_type = other DNA
gggtagggtggtgagatgaaatctctagggtgatcgttct

SEQ ID NO: 7 - DNA, 39 bp, synthetic construct
  misc_feature 1..39: note = Description of sequence: aptamer
  source 1..39: mol_type = other DNA
aggggctctagggtgggtaggatggtgagaccgcagcgt

SEQ ID NO: 8 - DNA, 40 bp, synthetic construct
  misc_feature 1..40: note = Description of sequence: aptamer
  source 1..40: mol_type = other DNA
cgtggggatgggtgggtggaggagcgatgatggcctacga

SEQ ID NO: 9 - DNA, 40 bp, synthetic construct; identical to SEQ ID NO: 1
  misc_feature 1..40: note = Description of sequence: aptamer
  source 1..40: mol_type = other DNA

SEQ ID NO: 10 - DNA, 15 bp, synthetic construct
  misc_feature 1..15: note = Description of sequence: aptamer
  source 1..15: mol_type = other DNA
ggttggtgtggttgg

SEQ ID NO: 11 - DNA, 29 bp, synthetic construct
  misc_feature 1..29: note = Description of sequence: aptamer
  source 1..29: mol_type = other DNA
agtccgtggtagggcaggttggggtgact

SEQ ID NO: 12 - DNA, 86 bp, synthetic construct
  misc_feature 1..86: note = Description of sequence: aptamer
  source 1..86: mol_type = other DNA
  variation 24..64: note = n is a, c, g, or t
tagggaagagaaggacatatgatnnnnnnnnnnnnnnnnnnnnnnnnnnnnnnnnnnnnnnnnttgactagtacatgaccacttga

SEQ ID NO: 13 - skipped

SEQ ID NO: 14 - DNA, 14 bp, synthetic construct
  misc_feature 1..14: note = Description of sequence: aptamer
  source 1..14: mol_type = other DNA
ggttgggaggttgg

SEQ ID NO: 15 - DNA, 15 bp, synthetic construct
  misc_feature 1..15: note = Description of sequence: aptamer
  source 1..15: mol_type = other DNA
ggttgggggggttgg

SEQ ID NO: 16 - DNA, 40 bp, synthetic construct; identical to SEQ ID NO: 2
  misc_feature 1..40: note = Description of sequence: aptamer
  source 1..40: mol_type = other DNA
  modified_base 40: mod_base = OTHER; note = nucleotide is covalently bound to hexaethylene glycol

SEQ ID NO: 17 - DNA, 40 bp, synthetic construct; identical to SEQ ID NO: 2
  misc_feature 1..40: note = Description of sequence: aptamer
  source 1..40: mol_type = other DNA
  modified_base 40: mod_base = OTHER; note = nucleotide is covalently bound to triethylene glycol

SEQ ID NO: 18 - DNA, 40 bp, synthetic construct; identical to SEQ ID NO: 2
  misc_feature 1..40: note = Description of sequence: aptamer
  source 1..40: mol_type = other DNA
  modified_base 40: mod_base = OTHER; note = nucleotide is covalently bound to cholesterol triethylene glycol

SEQ ID NO: 19 - DNA, 40 bp, synthetic construct; identical to SEQ ID NO: 4
  misc_feature 1..40: note = Description of sequence: aptamer
  source 1..40: mol_type = other DNA
  modified_base 40: mod_base = OTHER; note = nucleotide is covalently bound to hexaethylene glycol

SEQ ID NO: 20 - DNA, 40 bp, synthetic construct; identical to SEQ ID NO: 4
  misc_feature 1..40: note = Description of sequence: aptamer
  source 1..40: mol_type = other DNA
  modified_base 40: mod_base = OTHER; note = nucleotide is covalently bound to triethylene glycol

SEQ ID NO: 21 - DNA, 40 bp, synthetic construct; identical to SEQ ID NO: 4
  misc_feature 1..40: note = Description of sequence: aptamer
  source 1..40: mol_type = other DNA
  modified_base 40: mod_base = OTHER; note = nucleotide is covalently bound to cholesterol triethylene glycol

SEQ ID NO: 22 - DNA, 40 bp, synthetic construct
  misc_feature 1..40: note = Description of sequence: aptamer
  source 1..40: mol_type = other DNA
gcgcggtcccgatttggtgtaaaattccctcagccctaca

SEQ ID NO: 23 - DNA, 40 bp, synthetic construct
  misc_feature 1..40: note = Description of sequence: aptamer
  source 1..40: mol_type = other DNA
gcgattgcttcctccaacctcccaaccccagcaacaccgc

SEQ ID NO: 24 - DNA, 40 bp, synthetic construct
  misc_feature 1..40: note = Description of sequence: aptamer
  source 1..40: mol_type = other DNA
ttcaacgtcaacacccacccccccaaccgtaccttgacga